(19) United States
(12) Patent Application Publication
(10) Pub. No.: US 2010/0249302 A1
(43) Pub. Date: Sep. 30, 2010

(54) LIQUID COMPOSITION CONTAINING PERFLUORO-CHLOROETHER SOLVENT

(75) Inventors: Hiroki SUGIURA, Minami-Ashigara-shi (JP); Masayuki HARADA, Minami-Ashigara-shi (JP); Takayuki ITO, Haibara-gun (JP)

Correspondence Address: SUGHRUE-265550, 2100 PENNSYLVANIA AVE. NW, WASHINGTON, DC, 20037-3213, US

(73) Assignee: FUJIFILM CORPORATION, Tokyo (JP)

(21) Appl. No.: 12/749,909

(22) Filed: Mar. 30, 2010

(30) Foreign Application Priority Data
Mar. 30, 2009 (JP) 2009-083789
Mar. 29, 2010 (JP) 2010-076452

(51) Int. Cl. C09D 5/00; C07C 43/12

(52) U.S. Cl. 524/366; 568/684; 106/287.23

(57) ABSTRACT

Provided is a liquid composition including a perfluorochloroether solvent that contains a perfluorochloroether compound having a (chlorine content/fluorine content) value within the range of from 1.2 to 4.5; and an organic compound that has a fluorine content of from 0 to 79% by weight and that is dissolved in the perfluorochloroether solvent, wherein the (chlorine content/fluorine content) value is a value defined by the following formula:

(chlorine content/fluorine content)value=(number of chlorine atoms in perfluorochloroether×atomic weight of chlorine atom)/(number of fluorine atoms in perfluorochloroether×atomic weight of fluorine atom).

### BACKGROUND OF THE INVENTION

1. Field of the Invention

The present invention relates to a liquid composition containing a perfluorochloroether solvent, use of the liquid composition, and novel perfluorochloroether compounds.

2. Description of the Related Art

Perfluoro solvents have the characteristic properties such as incombustible properties, water-resistant properties, oil-resistant properties, and volatile properties and are inert to many reactive agents, therefore being used in various fields such as reaction solvents (see, for example, JP-A-5-59101 (the term “JP-A” as used herein means an “unexamined published Japanese patent application”)), solvents for a magnetic disc lubricant (see, for example, JP-A-2006-307123), solvents for a liquid toner (see, for example, JP-A-6-222623), and solvents in cosmetic products for preventing transfer (see, for example, JP-A-11-263709). However, while fluorine-containing organic compounds generally have a high solubility in perfluoro solvents, fluorine-free organic solvents have such a low solubility in perluoro solvents that they cause layer separation. Thus, there has been the problem that their application is limited to those substrates which have a high fluorine content.

In order to enhance solubility of a fluorine-free organic compound in a perfluoro solvent, 1,1,2-trichloro-1,2,2-trifluoroethane (CCl₂FCClF₂) which is a perfluoro solvent into which chlorine atoms are introduced is known as a solvent that can well dissolve both fluorine-containing organic compounds and fluorine-free organic compounds, and liquid compositions containing the solvent are used in, for example, detergents (see, for example, JP-A-5-4077), liquid phase fluorination reaction (see, for example, WO00/056694), and in polymerization reaction (see, for example, JP-T-2005-532413 (the term “JP-T” as used herein means an “unexamined published international patent application in Japanese”)). However, since this compound is a specified chlorofluorocarbon, its use is extremely strictly restricted. Also, it involves an additional problem that, since it has a boiling point as low as 48° C., it is not adequate for use at high temperatures.

As chlorine-containing perfluoro solvents which are not the specified chlorofluorocarbons, CF₂[OCF(CF₂Cl)₂]₂, ClCF₂CF[OCF(CF₂Cl)₂]₂ (see, for example, JP-T-4-500520), and CF₂ClCFClCOOCF₂CFClCF₂Cl (see, for example, JP-A-2006-28023) are disclosed as reaction solvents for liquid phase fluorination. However, although these solvents have a high dissolution capacity for fluorine-containing organic compounds, they do not have a very high dissolution capacity for fluorine-free organic compounds and, therefore, involve the problem that they are insufficient with respect to substrate-versatile properties. Thus, there has been desired to develop a perfluoro solvent that can well dissolve both fluorine-containing organic compounds and fluorine-free organic compounds.

### SUMMARY OF THE INVENTION

An object of the present invention is to provide a liquid composition which solves the above-described problem and which contains a perfluorochloroether solvent having an extremely high dissolution capacity for both fluorine-free organic compounds and fluorine-containing organic compounds.

In investigating perfluoro solvents, the inventors have found that the solubility of a fluorine-free organic compound in a perfluoro solvent closely correlates to the (chlorine content/fluorine content) value of a perfluorochloroether solvent, and that the dissolution capacity of the solvent for both a fluorine-free organic compound and a fluorine-containing compound can be enhanced to an extremely high level by adjusting the value to an appropriate range, thus having completed the invention. That is, the above-described problem can be solved by the following means.

1. A liquid composition comprising: a perfluorochloroether solvent that contains a perfluorochloroether compound having a (chlorine content/fluorine content) value within the range of from 1.2 to 4.5; and an organic compound that has a fluorine content of from 0 to 79% by weight and that is dissolved in the perfluorochloroether solvent, wherein the (chlorine content/fluorine content) value is a value defined by the following formula:

(chlorine content/fluorine content)value=(number of chlorine atoms in perfluorochloroether×atomic weight of chlorine atom)/(number of fluorine atoms in perfluorochloroether×atomic weight of fluorine atom).

2. The liquid composition according 1 described above, wherein the (chlorine content/fluorine content) value of the perfluorochloroether compound is within the range of 1.8 to 4.5.
3. The liquid composition according to 1 or 2 described above, wherein the perfluorochloroether solvent has a boiling point within the range of from 60° C. to 300° C. at a pressure of 760 mmHg.
4. The liquid composition according to any one of 1 to 3 described above, wherein the organic compound having a fluorine content of from 0 to 79% by weight is a polymerizable compound.
5. The liquid composition according to any one of 1 to 4 described above, wherein the organic compound having a fluorine content of from 0 to 79% by weight is a polymer.
6. The liquid composition according to any one of 1 to 5 described above, wherein the perfluorochloroether compound is a compound represented by the following general formula (1):

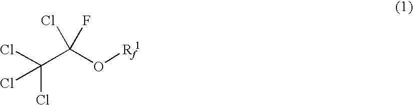

wherein R_{f}¹ represents a hydrocarbon group, all the hydrogen atoms of which are replaced by a fluorine atom or a chlorine atom, provided that the hydrocarbon group may contain an etheric oxygen atom.
7. The liquid composition according to 6 described above, wherein

the compound represented by the foregoing general formula (1) is a compound represented by the following formula (2), (3), (4) or (5):

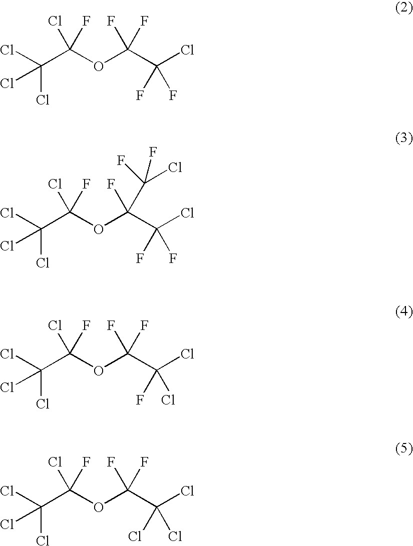

8. The liquid composition according to any one of 1 to 5 described above, wherein the perfluorochloroether compound is a compound represented by the following general formula (1′):

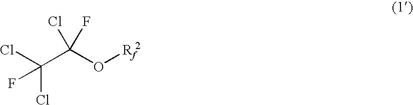

wherein R_{f}² represents a hydrocarbon group, all the hydrogen atoms of which are replaced by a fluorine atom or a chlorine atom, provided that the hydrocarbon group may contain an etheric oxygen atom.

9. The liquid composition according to 8 described above, wherein

the compound represented by the foregoing general formula (1′) is a compound represented by the following formula (2′), (3′), (4′) or (5′):

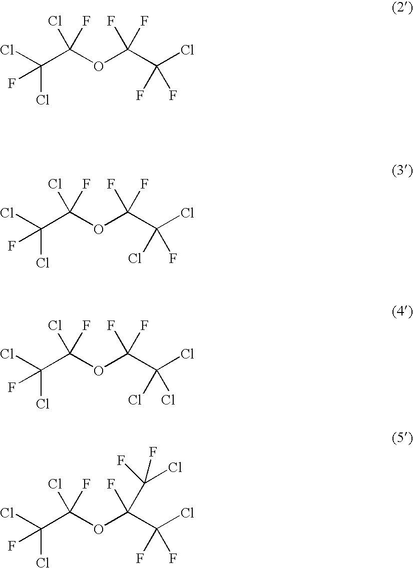

10. A liquid composition for coating, which comprises the liquid composition described in any one of 1 to 9 described above.
11. A chemical reaction solution which comprises the liquid composition described in any one of 1 to 9 described above.
12. A perfluorochloroether compound represented by the following formula (2):

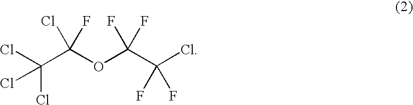

13. A perfluorochloroether compound represented by the following formula (3):

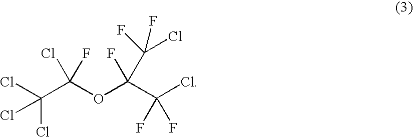

14. A perfluorochloroether compound represented by the following formula (4):

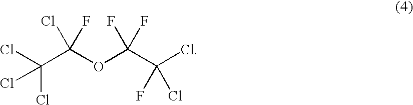

15. A perfluorochloroether compound represented by the following formula (5):

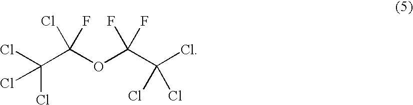

16. A perfluorochloroether compound represented by the following formula (2′):

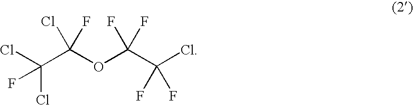

17. A perfluorochloroether compound represented by the following formula (3′):

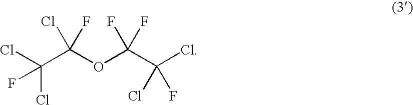

18. A perfluorochloroether compound represented by the following formula (4′):

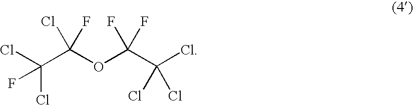

19. A perfluorochloroether compound represented by the following formula (5′):

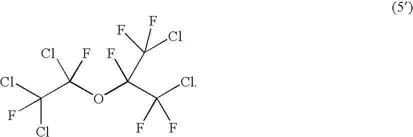

### DETAILED DESCRIPTION OF THE INVENTION

The liquid composition of the present invention containing the perfluorochloroether solvent (also referred to as a perluorochloroether-containing composition) is a liquid, perfluorochloroether-containing composition comprising a perfluorochloroether solvent containing a perfluorochloroether compound having a (chlorine content/fluorine content) value within the range of from 1.2 to 4.5, which contains dissolved therein an organic compound having a fluorine content of from 0 to 79% by weight, provided that the (chlorine content/fluorine content) value is a value defined by the following formula:

(chlorine content/fluorine content)value=(number of chlorine atoms in perfluorochloroether×atomic weight of chlorine atom)/(number of fluorine atoms in perfluorochloroether×atomic weight of fluorine atom).

The invention will be described in more detail below.

Additionally, the term “perfluorinated” as used herein in the invention means that all hydrogen atoms are replaced by fluorine atoms. Also, the term “perfluorochloroether” as used herein means ether wherein all hydrogen atoms are replaced by fluorine atoms or chlorine atoms.

The fluorine content in the invention is a value defined by the following formula:

fluorine content (% by weight)={(number of fluorine atoms in a compound×atomic weight of fluorine atom)/(molecular weight of the compound)}×100

(Perfluorochloroether Solvent)

The perfluorochloroether solvent in the invention is a solvent which contains at least one kind of perfluorochloroether compound having a (chlorine content/fluorine content) value within the range of from 1.2 to 4.5. In general, perfluoro solvents [for example, FC-72 (perfluorohexane); fluorine content: 79% by weight] has such an extremely low dissolution capacity for fluorine-free organic compounds that they are not uniformly mixed with fluorine-free organic compounds, causing layer separation due to the high fluorine content of the perfluoro solvents. Therefore, when the fluorine content of the perfluoro solvent is adjusted to an appropriate range, there can be obtained a solvent which has a high dissolution capacity for both fluorine-free organic compounds and fluorine-containing organic compounds. In the invention, it has been found that introduction of chlorine atoms is effective for adjusting the fluorine content of a perfluoro solvent to an appropriate range and, further, that a (chlorine content/fluorine content) value is an appropriate parameter of the dissolution capacity of the solvent for both fluorine-free organic compounds and fluorine-containing organic compounds, and it has been demonstrated that perfluorochloroether solvents having the value within the range of from 1.2 to 4.5 have an extremely high dissolution capacity for both fluorine-free organic compounds and fluorine-containing organic compounds. In case when the value is less than 1.2, the dissolution capacity for fluorine-free organic compounds becomes low though the dissolution capacity for fluorine-containing organic compounds becomes high, thus such solvents not being preferred. Also, in case when the value is more than 4.5, the dissolution capacity for fluorine-containing organic compounds becomes low though the dissolution capacity for fluorine-free organic compounds becomes high, thus such solvents not being preferred.

From the standpoint of enhancing solubility of a fluorine-free organic compound, the (chlorine content/fluorine content) value of a perfluorochloroether solvent is preferably within the range of from 1.8 to 4.5.

As perfluorochloroether compounds having the (chlorine content/fluorine content) value within the range of from 1.2 to 4.5, those compounds are preferred which can dissolve an organic compound of 0% by weight in fluorine content in an amount of 20% by weight or more and can dissolve an organic compound of 79% by weight in fluorine content in an amount of 20% by weight or more.

The perfluorochloroether solvent is not particularly limited as to its structure as long as it is liquid at a temperature under which it is used, and may be of any of straight-chain, branched, and cyclic structures. Also, the solvent is not particularly limited as to the position of chlorine atom and the number of etheric oxygen atoms.

Also, the perfluorochloroether solvent may be composed of one kind of perfluorochloroether compound, or may be a mixture of two or more kinds of perfluorochloroether compounds. In the case of containing two or more kinds of the perfluoroether compounds, it suffices that at least one kind of the perfluorochloroether compounds has the (chlorine content/fluorine content) value within the range of from 1.2 to 4.5. In addition, the perfluorochloroether solvent may contain other compounds as long as the object and advantage of the invention are attained. The proportion of the other compounds in the perfluorochloroether solvent is not particularly limited, and can be properly changed according to the purpose of use.

The perfluorochloroether solvents of the invention are not particularly limited as to boiling point, since it can properly be altered according to the use, but those solvents are preferred which have a boiling point of from 60° C. to 300° C. at the pressure of 760 mmHg. For example, in the case of using the perfluorochloroether-containing composition as a coating agent, the boiling point is preferably from 60° C. to 250° C., more preferably from 60° C. to 200° C., particularly preferably from 60° C. to 150° C., since too high a boiling point makes complicated the drying step after coating. Also, in the case of using the perfluorochloroether-containing composition as, for example, a chemical reaction solution or a chemical reaction solvent, it is possible to select and use an optimal solvent from the perfluorochloroether solvents having a boiling point of from 60° C. to 300° C. in consideration of reaction temperature and separability from the reaction substrate and from the reaction product.

Process for producing the perfluorochloroether compounds having the (chlorine content/fluorine content) value within the range of from 1.2 to 4.5 are not particularly limited, and the perfluorochloroether compounds can be produced according to various known processes. For example, they can be produced according to, for example, the production process described in Journal of Fluorine Chemistry, vol. 13, pp. 123-140 (1979), by chlorinating a chlorofluoroether compound as shown by the following scheme.

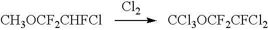

As an alternative process, the perfluorochloroethers can be produced according to, for example, the production process described in U.S. Pat. No. 2,803,666, by etherifying chloral, dehydrochlorinating the product, and then acting chlorine and antimony trifluoride on the product.

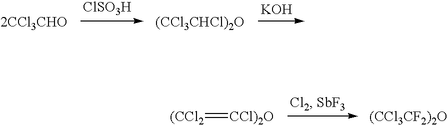

As the perfluorochloroether compounds, those compounds are preferred which are represented by the following general formula (1).

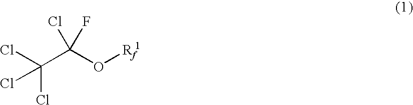

In the general formula (1), R_{f}¹ represents a hydrocarbon group, all the hydrogen atoms of which are replaced by a fluorine atom or a chlorine atom, (provided that the hydrocarbon group may contain an etheric oxygen atom), provided that the compound represented by the general formula (1) has a (chlorine content/fluorine content) value within the range of from 1.2 to 4.5.

R_{f}¹ may be of any of straight-chain, branched, and cyclic structures. R_{f}¹ is preferably an aliphatic hydrocarbon group, all of the hydrogen atoms of which are replaced by a fluorine atom or a chlorine atom, provided that the hydrocarbon group may contain an etheric oxygen atom, more preferably a saturated aliphatic hydrocarbon group, all of the hydrogen atoms of which are replaced by a fluorine atom or a chlorine atom, provided that the hydrocarbon group may contain an etheric oxygen atom.

R_{f}¹ represents a hydrocarbon group, all the hydrogen atoms of which are replaced by a fluorine atom or a chlorine atom, and the hydrocarbon group may contain an etheric oxygen atom but, from the standpoint of ease of production, the hydrocarbon group preferably does not contain any etheric oxygen atom.

R_{f}¹ contains preferably from 1 to 10 carbon atoms, more preferably from 1 to 5 carbon atoms, particularly preferably from 1 to 3 carbon atoms, most preferably 2 or 3 carbon atoms.

Specific examples of R_{f}¹ include a trifluoromethyl group, a chlorodifluoromethyl group, a pentafluoroethyl group, a 1-chloro-1,2,2,2-tetrafluoroethyl group, a 2-chloro-1,1,2,2-tetrafluoroethyl group, a 1,1-dichloro-2,2,2-trifluoroethyl group, a 1,2-dichloro-1,2,2-trifluoroethyl group, a 2,2-dichloro-1,1,2-trifluoroethyl group, a 2,2,2-trichloro-1,1-difluoroethyl group, a 1,1,2-trichloro-2,2-difluoroethyl group, a 1,2,2-trichloro-1,2-difluoroethyl group, a 1-chloro-1,2,2,3,3,3-hexafluoro-n-propyl group, a 2-chloro-1,1,2,3,3,3-hexafluoro-n-propyl group, a 3-chloro-1,1,2,3,3,3-hexafluoro-n-propyl group, a 1,1-dichloro-2,2,3,3,3-pentafluoro-n-propyl group, a 2,2-dichloro-1,1,3,3,3-pentafluoro-n-propyl group, a 3,3-dichloro-1,1,2,2,3-pentafluoro-n-propyl group, a 1,2-dichloro-1,2,3,3,3-pentafluoro-n-propyl group, a 1,3-dichloro-1,2,2,3,3-pentafluoro-n-propyl group, a 2,3-dichloro-1,1,2,3,3-pentafluoro-n-propyl group, a 1,1,2-trichloro-2,3,3,3-tetrafluoro-n-propyl group, a 1,1,3-trichloro-2,2,3,3-tetrafluoro-n-propyl group, a 1,2,2-trichloro-1,3,3,3-tetrafluoro-n-propyl group, a 1,2,3-trichloro-1,2,3,3-tetrafluoro-n-propyl group, a 2,2,3-trichloro-1,1,3,3-tetrafluoro-n-propyl group, a 2,3,3-trichloro-1,1,2,3-tetrafluoro-n-propyl group, a 3,3,3-trichloro-1,1,2,2-tetrafluoro-n-propyl group, a 1,1,2,2-tetrachloro-3,3,3-trifluoro-n-propyl group, a 1,1,2,3-tetrachloro-2,3,3-trifluoro-n-propyl group, a 1,1,3,3-tetrachloro-2,2,3-trifluoro-n-propyl group, a 1,2,2,3-tetrachloro-1,3,3-trifluoro-n-propyl group, a 1,2,3,3-tetrachloro-1,2,3-trifluoro-n-propyl group, a 1,3,3,3-tetrachloro-1,2,2-trifluoro-n-propyl group, a 2,2,3,3-tetrachloro-1,1,3-trifluoro-n-propyl group, a 2,3,3,3-tetrachloro-1,1,2-trifluoro-n-propyl group, a 1-chloro-1,2,2,2′,2′,2′-hexafluoro-i-propyl group, a 2-chloro-1,2,2,2′,2′,2′-hexafluoro-i-propyl group, a 1,2-dichloro-2,2,2′,2′,2′-pentafluoro-i-propyl group, a 2,2-dichloro-1,2,2′,2′,2′-pentafluoro-i-propyl group, a 2,2′-dichloro-1,2,2,2′,2′-pentafluoro-i-propyl group, a 1,2,2-trichloro-2,2′,2′,2′-tetrafluoro-i-propyl group, a 1,2,2′-trichloro-2,2,2′,2′-tetrafluoro-i-propyl group, a 2,2,2-trichloro-1,2′,2′,2′-tetrafluoro-i-propyl group, a 2,2,2′-trichloro-1,2,2′,2′-tetrafluoro-i-propyl group, a 1,2,2,2-tetrachloro-2′,2′,2′-trifluoro-i-propyl group, a 1,2,2,2′-tetrachloro-2,2′,2′-trifluoro-i-propyl group, a 2,2,2,2′-tetrachloro-1,2′,2′-trifluoro-i-propyl group, a 2,2,2′,2′-tetrachloro-1,2,2′-trifluoro-i-propyl group, a 1-chloro-2,2,3,3-tetrafluorocyclopropyl group, a 2-chloro-1,2,3,3-tetraflorocyclopropyl group, a 1,2-dichloro-2,3,3-trifluorocyclopropyl group, a 2,2-dichloro-1,3,3-trifluorocyclopropyl group, a 2,3-dichloro-1,2,3-trifluorocyclopropyl group, a 1,2,3-trichloro-2,3-difluorocyclopropyl group, a 1,2,2-trichloro-3,3-difluorocyclopropyl group, and a 2,2,3-trichloro-1,3-difluorocyclopropyl group.

R_{f} is preferably a trifluoromethyl group, a pentafluoroethyl group, a 2-chloro-1,1,2,2-tetrafluoroethyl group, a 2,2-dichloro-1,1,2-trifluoroethyl group, a 2,2,2-trichloro-1,1-difluoroethyl group, a 2-chloro-1,1,2,3,3,3-hexafluoro-n-propyl group, a 3-chloro-1,1,2,2,3,3-hexafluoro-n-propyl group, a 2,3-dichloro-1,1,2,3,3-pentafluoro-n-propyl group, a 2,2-dichloro-1,1,3,3,3-pentafluoro-n-propyl group, a 3,3-dichloro-1,1,2,2,3-pentafluoro-n-propyl group, a 2,2,3-trichloro-1,1,3,3-tetrafluoro-n-propyl group, a 2,3,3-trichloro-1,1,2,3-tetrafluoro-n-propyl group, a 3,3,3-trichloro-1,1,2,2-tetrafluoro-n-propyl group, a 2,2,3,3-tetrachloro-1,1,3-trifluoro-n-propyl group, a 2,3,3,3-tetrachloro-1,1,2-trifluoro-n-propyl group, a 2-chloro-1,2,2,2′,2′,2′-hexafluoro-i-propyl group, a 2,2-dichloro-1,2,2′,2′,2′-pentafluoro-i-propyl group, a 2,2′-dichloro-1,2,2,2′,2′-pentafluoro-i-propyl group, a 2,2,2-trichloro-1,2′,2′,2′-tetrafluoro-i-propyl group, a 2,2,2′-trichloro-1,2,2′,2′-tetrafluoro-i-propyl group, a 2,2,2,2′-tetrachloro-1,2′,2′-trifluoro-i-propyl group, a 2,2,2′,2′-tetrachloro-1,2,2′-trifluoro-i-propyl group, a pentafluorocyclopropyl group, a 2-chloro-1,2,3,3-tetrafluorocyclopropyl group, a 2,2-dichloro-1,3,3-trifluorocyclopropyl group, a 2,3-dichloro-1,2,3-trifluorocyclopropyl group, or a 2,2,3-trichloro-1,3-difluorocyclopropyl group, more preferably a trifluoromethyl group, a 2-chloro-1,1,2,2-tetrafluoroethyl group, a 2,2-dichloro-1,1,2-trifluoroethyl group, a 2,2,2-trichloro-1,1-difluoroethyl group, a 2,3-dichloro-1,1,2,3,3-pentafluoro-n-propyl group, a 3,3-dichloro-1,1,2,2,3-pentafluoro-n-propyl group, a 3,3,3-trichloro-1,1,2,2-tetrafluoro-n-propyl group, a 2,2-dichloro-1,2,2′,2′,2′-pentafluoro-i-propyl group, a 2,2′-dichloro-1,2,2,2′,2′-pentafluoro-i-propyl group, a 2,2,2-trichloro-1,2′,2′,2′-tetrafluoro-i-propyl group, a 2,2,2′-trichloro-1,2,2′,2′-tetrafluoro-i-propyl group, a 2,2,2,2′-tetrachloro-1,2′,2′-trifluoro-i-propyl group, a 2,2,2′,2′-tetrachloro-1,2,2′-trifluoro-i-propyl group, a 2-chloro-1,2,3,3-tetrafluorocyclopropyl group, a 2,2-dichloro-1,3,3-trifluorocyclopropyl group, a 2,3-dichloro-1,2,3-trifluorocyclopropyl group, or a 2,2,3-trichloro-1,3-difluorocyclopropyl group, particularly preferably a trifluoromethyl group, a 2-chloro-1,1,2,2-tetrafluoroethyl group, a 2,2-dichloro-1,1,2-trifluoroethyl group, 2,2,2-trichloro-1,1-difluoroethyl group, 2,2′-dichloro-1,2,2,2′,2′-pentafluoro-i-propyl group, or a 2,3-dichloro-1,1,2,3,3-pentafluoro-n-propyl group.

Specific examples of the compound represented by the general formula (1) are shown below which, however, do not limit the invention in any way.

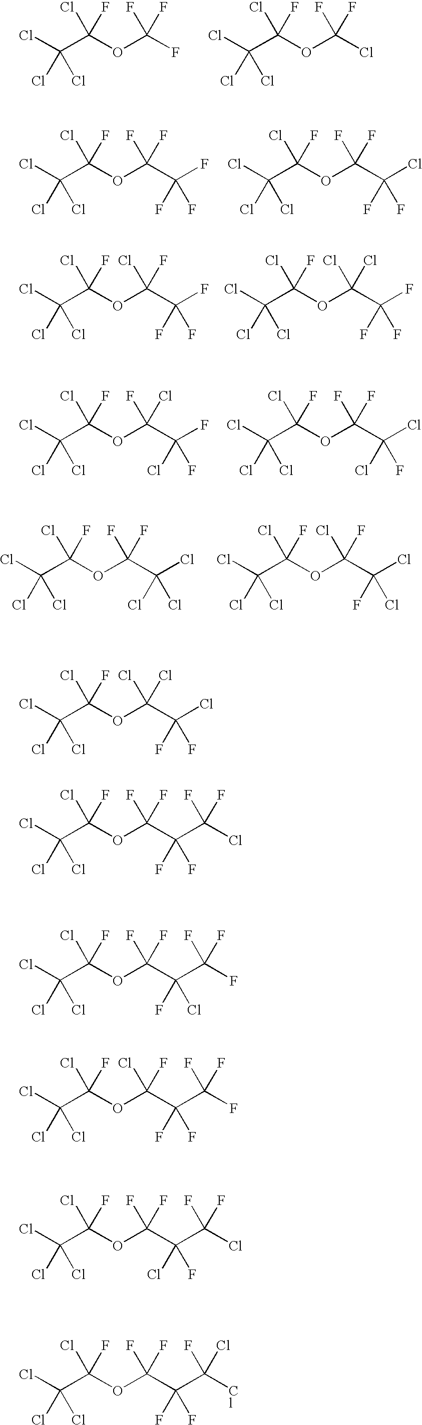

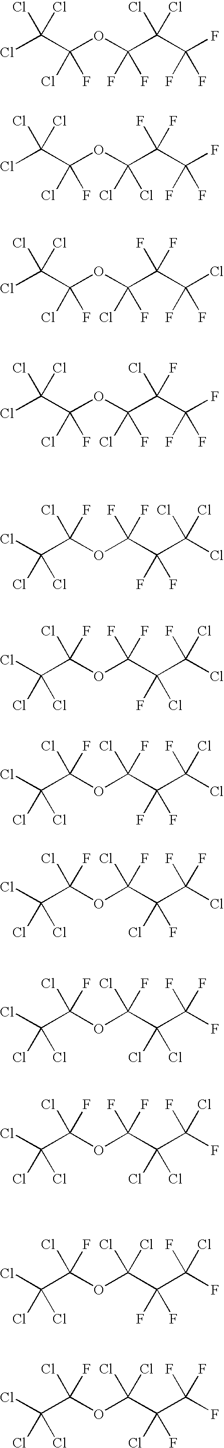

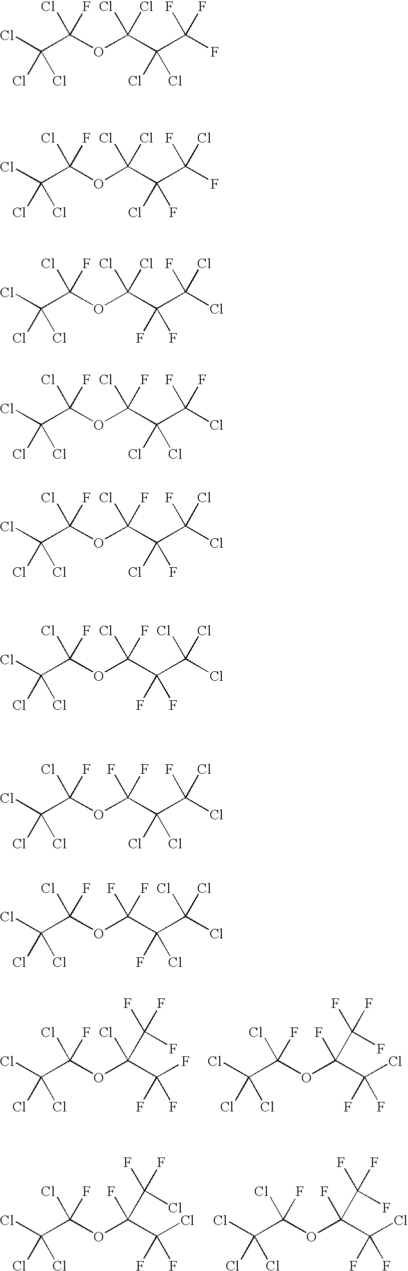

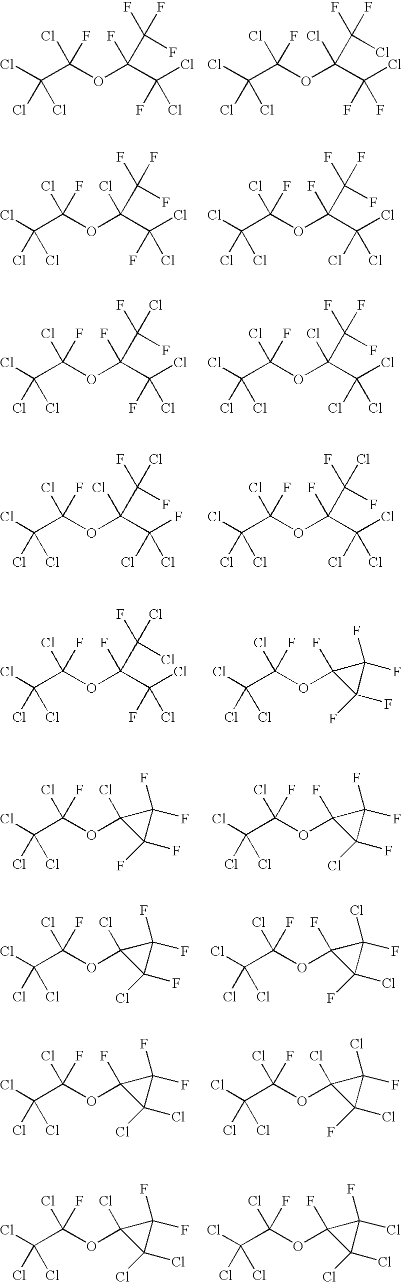

Of the above-shown compounds, compounds represented by the following formulae (2), (3), (4), and (5) are more preferred for the reason that starting materials for the compounds are easily available.

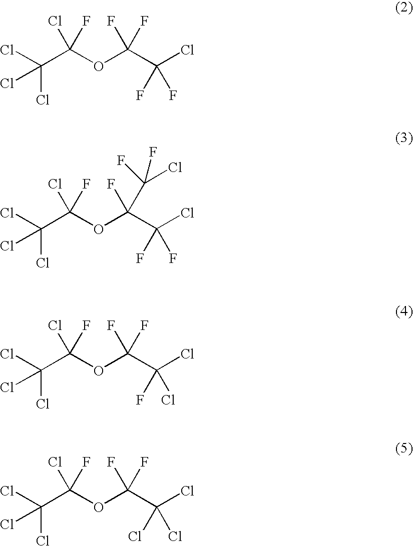

Also, as the perfluorochloro ether compounds, those compounds which are represented by the following general formula (1′) are preferred in addition to the compounds represented by the foregoing general formula (1):

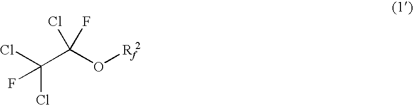

In the general formula (1′), R_{f}² represents a hydrocarbon group, all the hydrogen atoms of which are replaced by a fluorine atom or a chlorine atom (provided that the hydrocarbon group may contain an etheric oxygen atom), provided that the compound represented by the general formula (1′) has a (chlorine content/fluorine content) value within the range of from 1.2 to 4.5.

R_{f}² may be of any of straight-chain, branched, and cyclic structures. R_{f}² is preferably an aliphatic hydrocarbon group, all of the hydrogen atoms of which are replaced by a fluorine atom or a chlorine atom, provided that the hydrocarbon group may contain an etheric oxygen atom, more preferably a saturated aliphatic hydrocarbon group, all of the hydrogen atoms of which are replaced by a fluorine atom or a chlorine atom, provided that the hydrocarbon group may contain an etheric oxygen atom.

R_{f}² represents a hydrocarbon group, all the hydrogen atoms of which are replaced by a fluorine atom or a chlorine atom, and the hydrocarbon group may contain an etheric oxygen atom but, from the standpoint of ease of production, the hydrocarbon group preferably does not contain any etheric oxygen atom. R_{f}² contains preferably from 1 to 10 carbon atoms, more preferably from 1 to 5 carbon atoms, particularly preferably from 1 to 3 carbon atoms, most preferably 2 or 3 carbon atoms.

Specific examples of R_{f}² include a chlorodifluoromethyl group, a dichlorofluoromethyl group, a 1-chloro-1,2,2,2-tetrafluoroethyl group, a 2-chloro-1,1,2,2-tetrafluoroethyl group, a 1,1-dichloro-2,2,2-trifluoroethyl group, a 1,2-dichloro-1,2,2-trifluoroethyl group, a 2,2-dichloro-1,1,2-trifluoroethyl group, a 2,2,2-trichloro-1,1-difluoroethyl group, a 1,1,2-trichloro-2,2-difluoroethyl group, a 1,2,2-trichloro-1,2-difluoroethyl group, a 1,1,2,2-tetrachloro-2-fluoroethyl group, a 1,2,2,2-tetrachloro-1-fluoroethyl group, a 1,1-dichloro-2,2,3,3,3-pentafluoro-n-propyl group, 2,2-dichloro-1,1,3,3,3-pentafluoro-n-propyl group, a 3,3-dichloro-1,1,2,2,3-pentafluoro-n-propyl group, a 1,2-dichloro-1,2,3,3,3-pentafluoro-n-propyl group, a 1,3-dichloro-1,2,2,3,3-pentafluoro-n-propyl group, a 2,3-dichloro-1,1,2,3,3-pentafluoro-n-propyl group, a 1,1,2-trichloro-2,3,3,3-tetrafluoro-n-propyl group, a 1,1,3-trichloro-2,2,3,3-tetrafluoro-n-propyl group, a 1,2,2-trichloro-1,3,3,3-tetrafluoro-n-propyl group, a 1,2,3-trichloro-1,2,3,3-tetrafluoro-n-propyl group, a 2,2,3-trichloro-1,1,3,3-tetrafluoro-n-propyl group, a 2,3,3-trichloro-1,1,2,3-tetrafluoro-n-propyl group, a 3,3,3-trichloro-1,1,2,2-tetrafluoro-n-propyl group, a 1,1,2,2-tetrachloro-3,3,3-trifluoro-n-propyl group, a 1,1,2,3-tetrachloro-2,3,3-trifluoro-n-propyl group, a 1,1,3,3-tetrachloro-2,2,3-trifluoro-n-propyl group, a 1,2,2,3-tetrachloro-1,3,3-trifluoro-n-propyl group, a 1,2,3,3-tetrachloro-1,2,3-trifluoro-n-propyl group, a 1,3,3,3-tetrachloro-1,2,2-trifluoro-n-propyl group, a 2,2,3,3-tetrachloro-1,1,3-trifluoro-n-propyl group, a 2,3,3,3-tetrachloro-1,1,2-trifluoro-n-propyl group, a 1,1,2,2,3-pentachloro-3,3-difluoro-n-propyl group, a 1,2,2,3,3-pentachloro-1,3-difluoro-n-propyl group, a 1,2,3,3,3-pentachloro-1,2-difluoro-n-propyl group, a 2,2,3,3,3-pentachloro-1,1-difluoro-n-propyl group, a 1,1,3,3,3-pentachloro-2,2-difluoro-n-propyl group, a 1,1,2,3,3-pentachloro-2,3-difluoro-n-propyl group, a 1,2-dichloro-2,2,2′,2′,2′-pentafluoro-i-propyl group, a 2,2-dichloro-1,2,2′,2′,2′-pentafluoro-i-propyl group, a 2,2′-dichloro-1,2,2,2′,2′-pentafluoro-i-propyl group, a 1,2,2-trichloro-2,2′,2′,2′-tetrafluoro-i-propyl group, a 1,2,2′-trichloro-2,2,2′,2′-tetrafluoro-i-propyl group, a 2,2,2-trichloro-1,2′,2′,2′-tetrafluoro-i-propyl group, a 2,2,2′-trichloro-1,2,2′,2′-tetrafluoro-i-propyl group, a 1,2,2,2-tetrachloro-2′,2′,2′-trifluoro-i-propyl group, a 1,2,2,2′-tetrachloro-2,2′,2′-trifluoro-i-propyl group, a 2,2,2,2′-tetrachloro-1,2′,2′-trifluoro-i-propyl group, a 2,2,2′,2′-tetrachloro-1,2,2′-trifluoro-i-propyl group, a 1,2,2,2,2′-pentachloro-2′,2′-difluoro-i-propyl group, a 1,2,2,2′,2′-pentachloro-2,2′-difluoro-i-propyl group, a 2,2,2,2′,2′-pentachloro-1,2′-difluoro-i-propyl group, a 1-chloro-2,2,3,3-tetrafluorocyclopropyl group, a 2-chloro-1,2,3,3-tetraflorocyclopropyl group, a 1,2-dichloro-2,3,3-trifluorocyclopropyl group, a 2,2-dichloro-1,3,3-trifluorocyclopropyl group, a 2,3-dichloro-1,2,3-trifluorocyclopropyl group, a 1,2,3-trichloro-2,3-difluorocyclopropyl group, a 1,2,2-trichloro-3,3-difluorocyclopropyl group, a 2,2,3-trichloro-1,3-difluorocyclopropyl group, a 1,2,2,3-tetrachloro-3-fluorocyclopropyl group, and a 2,2,3,3-tetrachloro-1-fluorocyclopropyl group.

R_{f}² is preferably a 2-chloro-1,1,2,2-tetrafluoroethyl group, a 2,2-dichloro-1,1,2-trifluoroethyl group, a 2,2,2-trichloro-1,1-difluoroethyl group, a 2,2-dichloro-1,1,3,3,3-pentafluoro-n-propyl group, a 3,3-dichloro-1,1,2,3,3-pentafluoro-n-propyl group, a 2,3-dichloro-1,1,2,3,3-pentafluoro-n-propyl group, a 2,2,3-trichloro-1,1,3,3-tetrafluoro-n-propyl group, a 2,3,3-trichloro-1,1,2,3-tetrafluoro-n-propyl group, a 3,3,3-trichloro-1,1,2,2-tetrafluoro-n-propyl group, a 2,2,3,3-tetrachloro-1,1,3-trifluoro-n-propyl group, a 2,3,3,3-tetrachloro-1,1,2-trifluoro-n-propyl group, a 2,2,3,3,3-pentachloro-1,1-difluoro-n-propyl group, a 2,2-dichloro-1,2,2′,2′,2′-pentafluoro-i-propyl group, a 2,2′-dichloro-1,2,2,2′,2′-pentafluoro-i-propyl group, a 2,2,2-trichloro-1,2∝,2′,2′-tetrafluoro-i-propyl group, a 2,2,2′-trichloro-1,2,2′,2′-tetrafluoro-i-propyl group, a 2,2,2,2′-tetrachloro-1,2′,2′-trifluoro-i-propyl group, a 2,2,2′,2′-tetrachloro-1,2,2′-trifluoro-i-propyl group, a 2,2,2,2′,2′-pentachloro-1,2′-difluoro-i-propyl group, a 2-chloro-1,2,3,3-tetrafluorocyclopropyl group, a 2,2-dichloro-1,3,3-trifluorocyclopropyl group, a 2,3-dichloro-1,2,3-trifluorocyclopropyl group, a 2,2,3-trichloro-1,3-difluorocyclopropyl group or a 2,2,3,3-tetrachloro-1-fluorocyclopropyl group, more preferably a 2-chloro-1,1,2,2-tetrafluoroethyl group, a 2,2-dichloro-1,1,2-trifluoroethyl group, a 2,2,2-trichloro-1,1-difluoroethyl group, a 3,3-dichloro-1,1,2,2,3-pentafluoro-n-propyl group, a 2,3-dichloro-1,1,2,3,3-pentafluoro-n-propyl group, a 3,3,3-trichloro-1,1,2,2-tetrafluoro-n-propyl group, a 2,2-dichloro-1,2,2′,2′,2′-pentafluoro-i-propyl group, a 2,2′-dichloro-1,2,2,2′,2′-pentafluoro-i-propyl group, a 2,2,2-trichloro-1,2′,2′,2′-tetrafluoro-i-propyl group, a 2,2,2′-trichloro-1,2,2′,2′-tetrafluoro-i-propyl group, a 2,2,2,2′-tetrachloro-1,2′,2′-trifluoro-i-propyl group, a 2,2,2′,2′-tetrachloro-1,2,2′-trifluoro-i-propyl group, a 2,2,2,2′,2′-pentachloro-1,2′-difluoro-i-propyl group, a 2-chloro-1,2,3,3-tetrafluorocyclopropyl group, a 2,2-dichloro-1,3,3-trifluorocyclopropyl group, a 2,3-dichloro-1,2,3-trifluorocyclopropyl group, a 2,2,3-trichloro-1,3-difluorocyclopropyl group, or a 2,2,3,3-tetrachloro-1-fluorocyclopropyl group, particularly preferably a 2-chloro-1,1,2,2-tetrafluoroethyl group, a 2,2-dichloro-1,1,2-trifluoroethyl group, 2,2,2-trichloro-1,1-difluoroethyl group, a 2,3-dichloro-1,1,2,3,3-pentafluoro-n-propyl group, or a 2,2′-dichloro-1,2,2,2′,2′-pentafluoro-1-propyl group.

Specific examples of the compound represented by the general formula (1′) are shown below which, however, do not limit the invention in any way.

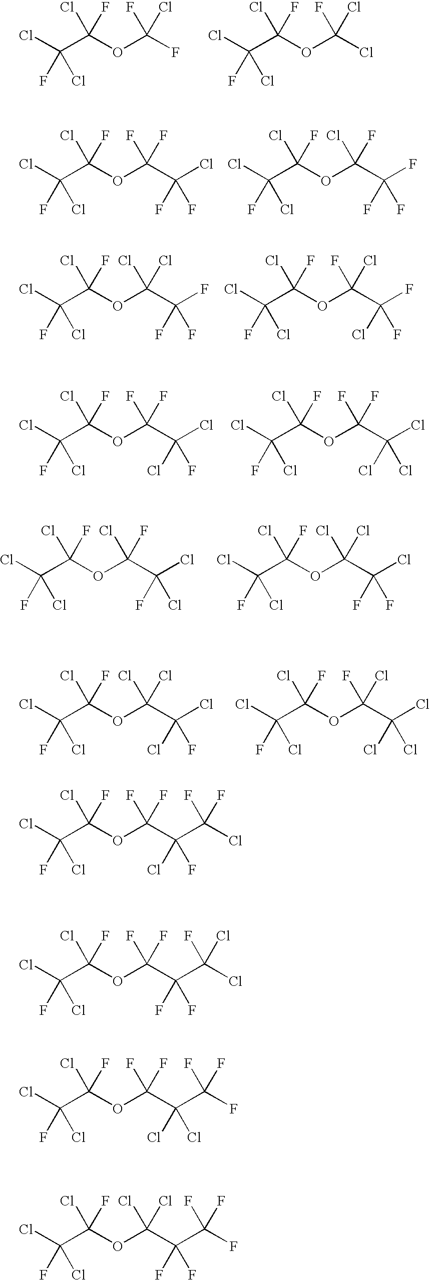

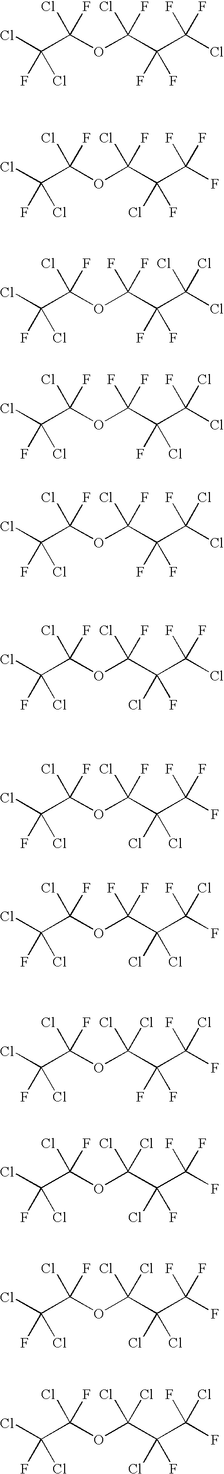

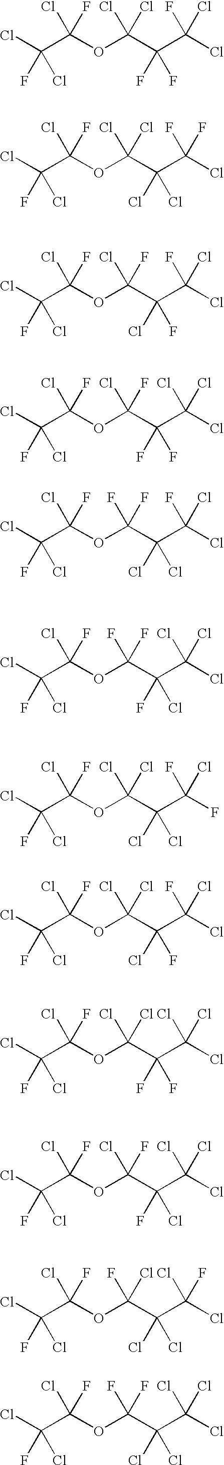

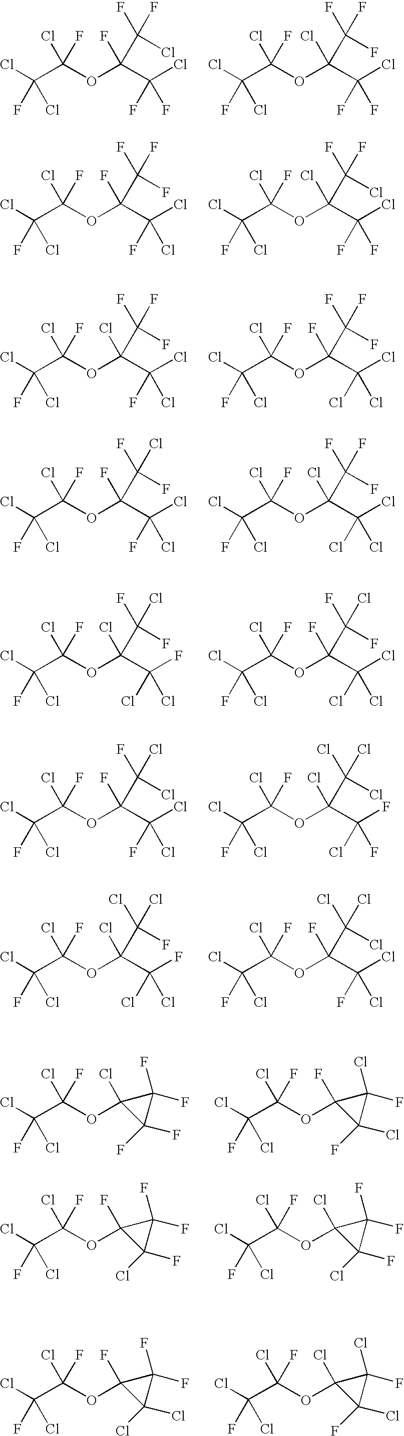

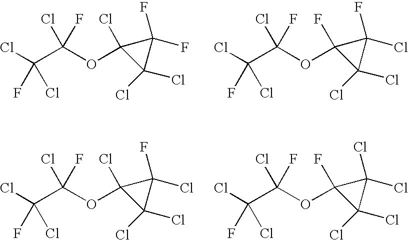

Of the above-shown compounds, as compounds represented by the formula (1′), compounds represented by the following formulae (2′), (3′), (4′), and (5′) are more preferred for the reason that starting materials for the compounds are easily available.

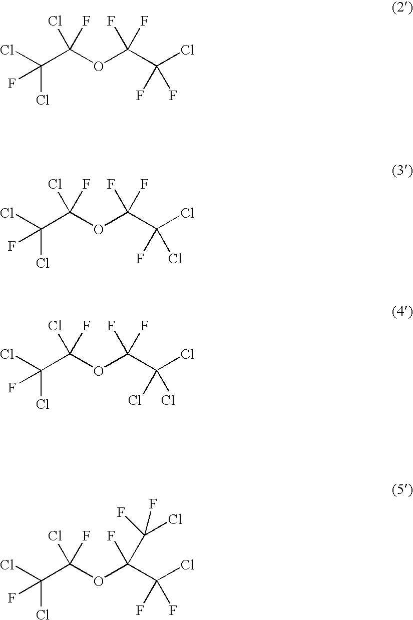

Processes for producing the compounds represented by the general formula (1) and the compounds represented by the general formula (1′) are not particularly limited, and the compounds can be produced according to various known processes. Compounds represented by the general formula (1) can be produced by, for example, perfluorinating a compound represented by the following general formula (8). The compounds represented by the general formula (8) can be produced by, for example, reacting chloral with an alcohol compound (6) to produce a compound (7) described below according to the production process described in U.S. Pat. No. 2,803,665 and Journal of Organometallic Chemistry, vol. 71, pp. 335-346 (1974), followed by chlorinating the product. In the following scheme, R represents a monovalent substituent that can be converted to R_{f}¹ by perfluorination.

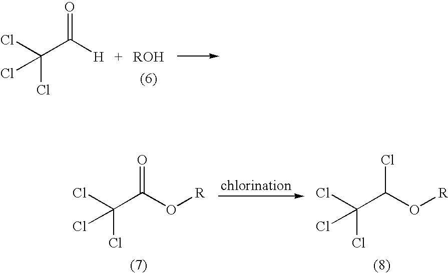

Processes for perfluorinating the compound represented by the general formula (8) are also not particularly limited, and the perfluorination can be conducted according to various known processes. For example, the compounds of the general formula (1) can be produced according to the process described in JP-T-4-500520 by reacting with a fluorine gas in a perfluoro solvent (liquid-phase fluorination reaction) as shown by the following scheme.

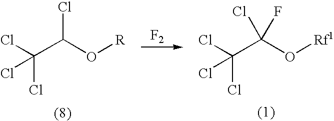

Compounds represented by the general formula (1′) can be produced by, for example, dehydrochlorinating a compound represented by the foregoing general formula (8) with a base to produce compound (6′), then perfluorinating the compound (6′) with a fluorine gas.

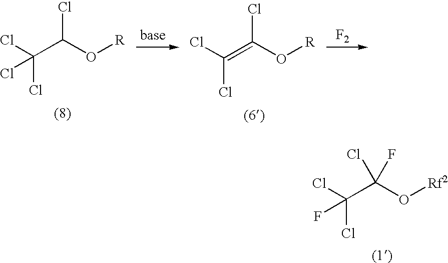

The perfluorochloroether solvent may be used as a mixture with other fluorine-containing solvent or fluorine-free solvent. In such cases, the content of the perfluorochloroether compound in the mixed solvent is not particularly limited, since the content can be properly changed depending upon solubility of the organic compound having a fluorine content of from 0 to 79% by weight and upon intended use. For example, in the case of dissolving both a fluorine-free organic compound and a fluorine-containing organic compound, the perfluorochloroether compound having a (chlorine content/fluorine content) value within the range of from 1.2 to 4.5 is contained in the perfluorochloroether solvent in a content of preferably from 1 to 100% by weight, more preferably from 10 to 100% by weight, particularly preferably from 20 to 100% by weight, in order to enhance solubility of both the fluorine-free organic compound and the fluorine-containing organic compound.

Fluorine-containing solvents which can be mixed with the perfluorochloroether solvent to use cannot particularly be limited, since they are properly changed depending upon solubility of the organic compound having a fluorine content of from 0 to 79% by weight and upon intended use, and examples thereof include fluorobenzene, difluorobenzene, benzotrifluoride, hexafluorobenzene, 2,2,2-trifluoroethanol, fluoroacetic acid, difluoroacetic acid, trifluoroacetic acid, hexafluoroacetone, 1,1,1,3,3,3-hexafluoro-2-propanol, perfluoroalkane compounds [e.g., FC-72 (trade name; manufactured by Sumitomo 3M), etc.], perfluoroether compounds [FC-75, FC-77 (both being trade names; manufactured by Sumitomo 3M), etc.], perfluoropolyether compounds [trade name: Krytox® (trademark of DuPont); trade name: Fomblin® (trademark of Solvay Solexis); trade name: Galden® (trademark of Solvay Solexis); trade name: Demnum; manufactured by Daikin Industries; etc.], hydrochlorofluorocarbon compounds [trade name: AK-225; manufactured by AGC; etc.], chlorofluorocarbon compounds [CFC-11, CFC-113, etc.], hydrofluorocarbon compounds [trade name: Vertrel® (trademark of Mitsui DuPont Fluorochemicals Co., Ltd.); etc.], hydrofluoroether compounds [trade name: Novec® (trademark of 3M); etc.], chlorofluoropolyether compounds, perfluorotrialkylamine compounds, and inert fluids [trade name: Fluorrinert® (trade name of 3M); trade name: Halocarbon (trademark of Halocarbon Co.); etc.], and these may be used as a mixture of two or more thereof.

The content of the fluorine-containing solvent in the perfluorochloroether solvent cannot be particularly limited, since the content can be properly changed depending upon kind of the fluorine-containing solvent, solubility of the organic compound having a fluorine content of from 0 to 79% by weight, and upon intended use. For example, in the case of dissolving both a fluorine-free organic compound and a fluorine-containing organic compound, the fluorine-containing solvent is contained in the perfluorochloroether solvent in a content of preferably from 0 to 99% by weight, more preferably from 0.1 to 99% by weight, still more preferably from 0.1 to 90% by weight, particularly preferably from 0.1 to 80% by weight, in order to enhance solubility of both the fluorine-free organic compound and the fluorine-containing organic compound.

Fluorine-free solvents which can be mixed with the perfluorochloroether solvent to use cannot particularly be limited, since they are properly altered according to the particular use, and examples thereof include alcohol series solvents such as methanol, ethanol, n-propanol, i-propanol, n-butanol, sec-butanol, tert-butanol, ethylene glycol, diethylene glycol, and benzyl alcohol; ketone series solvents such as acetone, 2-butanone, and cyclohexanone; nitrile series solvents such as acetonitrile, propionitrile, and benzonitrile; carboxylic acid series solvents such as formic acid, acetic acid, and propionic acid; hydrocarbon series solvents such as n-pentane, cyclopentane, n-hexane, cyclohexane, n-heptane, benzene, toluene, xylene, and mesitylene; halogen-containing solvents such as chloroform, carbon tetrachloride, dichloromethane, 1,2-dichloroethane, 1,1,2,2-tetrachloroethane, chlorobenzene, bromobenzene, iodobenzene, and dichlorobenzene; ether series solvents such as diethyl ether, diisopropyl ether, cyclopentyl methyl ether, 1,2-dimethoxyethane, diglyme, triglyme, tetrahydrofuran, tetrahydropyran, anisole, and diphenyl ether; thioether series solvents such as thioanisole and phenyl sulfide; amide series solvents such as N,N-dimethylformamide, N,N-dimethylacetamide, N-methyl-2-pyrrolidone, N-ethyl-2-pyrolidone, and N-butyl-2-pyrrolidone; amine series solvents such as triethylamine, diisopropylethylamine, piperidine, pyrrolidine, aniline, N,N-dimethylaniline, and morpholine; pyridine series solvents such as pyridine and 2,6-lutidine; dimethylsulfoxide; hexamethylphosphoric acid triamide; and hexamethylphosphorous acid triamide, and these may be used as a mixture of two or more thereof.

The content of the fluorine-free solvent in the perfluorochloroether solvent cannot be particularly limited, since the content can be properly changed depending upon kind of the fluorine-free solvent, solubility of the organic compound having a fluorine content of from 0 to 79% by weight, and upon intended use. For example, in the case of dissolving both a fluorine-free organic compound and a fluorine-containing organic compound, the fluorine-free solvent is contained in the perfluorochloroether solvent in a content of preferably from 0 to 99% by weight, more preferably from 0.1 to 99% by weight, still more preferably from 0.1 to 90% by weight, particularly preferably from 0.1 to 80% by weight, in order to enhance solubility of both the fluorine-free organic compound and the fluorine-containing organic compound.

(Organic Compounds Having a Fluorine Content of from 0 to 79% by Weight)

Organic compounds in the invention having a fluorine content of from 0 to 79% by weight are fluorine-free organic compounds and fluorine-containing organic compounds which have a fluorine content of 79% by weight or less. The organic compounds in the invention having a fluorine content of from 0 to 79% by weight are not particularly limited as long as they dissolve into the perfluorochloroether solvent in the invention, and may be in the form of any of liquid, solid, and gas. The perfluorochloroether-containing composition of the invention contains the organic compound having a fluorine content of from 0 to 79% by weight in a dissolved state and, as the organic compound, one kind or two or more kinds of the compounds may be used.

The term of organic compounds in the invention means compounds which have a parent structure of chain-like, branched, or cyclic hydrocarbon or compounds wherein part of carbon atoms, hydrocarbons, or hydrogen atoms constituting the parent structure are replaced by other atoms or substituents. The hydrocarbon may be saturated or unsaturated, and may be aromatic. The aromatic moiety may be a single ring or a condensed ring, and may be a hetero ring containing a hetero atom (e.g., nitrogen atom, sulfur atom, or oxygen atom) in the ring. The hetero ring may be a saturated ring or an unsaturated ring, and may be a single ring or a condensed ring. The organic compounds in the invention may be organometallic compounds or metal complexes. Also, the organic compounds of the invention may be cations or anions. In the case where the organic compounds are cations, the counter anion may be an organic anion or an inorganic anion and, in the case where the organic compounds are anions, the counter cation may be an organic cation or an inorganic cation.

Other atoms than carbon atom which the organic compounds in the invention can contain are not particularly limited, and include lithium, sodium, potassium, magnesium, scandium, yttrium, titanium, zirconium, hafnium, vanadium, chromium, molybdenum, tungsten, manganese, rhenium, iron, ruthenium, osmium, cobalt, rhodium, iridium, nickel, palladium, platinum, copper, silver, gold, zinc, cadmium, mercury, boron, aluminum, gallium, indium, silicon, germanium, tin, lead, nitrogen, phosphorus, oxygen, sulfur, selenium, tellurium, fluorine, chlorine, bromine, and iodine. Preferred examples thereof are lithium, sodium, potassium, magnesium, titanium, ruthenium, cobalt, rhodium, iridium, nickel, palladium, platinum, copper, silver, gold, zinc, boron, aluminum, gallium, silicon, tin, nitrogen, phosphorus, oxygen, sulfur, selenium, tellurium, fluorine, chlorine, bromine, and iodine, and more preferred examples thereof are lithium, sodium, potassium, magnesium, titanium, ruthenium, cobalt, rhodium, nickel, palladium, zinc, boron, aluminum, silicon, tin, nitrogen, phosphorus, oxygen, sulfur, selenium, fluorine, chlorine, bromine, and iodine.

Substituents which the organic compounds in the invention may have are not particularly limited, and include an alkyl group (e.g., a methyl group, an ethyl group, a propyl group, an isopropyl group, a tert-butyl group, a pentyl group, a hexyl group, an octyl group, a dodecyl group, a tridecyl group, a tetradecyl group, a pentadecyl group, etc.), a cycloalkyl group (e.g., a cyclopropyl group, a cyclopentyl group, a cyclohexyl group, etc.), an alkenyl group (e.g., a vinyl group, an allyl group, a butenyl group, an octenyl group, etc.), a cycloalkenyl group (e.g., a 2-cyclopenten-1-yl group, a 2-cyclohexen-1-yl group, etc.), an alkynyl group (e.g., a propargyl group, an ethynyl group, a trimethylsilylethynyl group, etc.), a halogen atom (e.g., fluorine, chlorine, bromine, iodine, etc.), an aryl group (e.g., a phenyl group, a naphthyl group, a p-tolyl group, a m-chlorophenyl group, etc.), a heteroaryl group (e.g., a pyridyl group, a thiazolyl group, an oxazolyl group, an imidazolyl group, a furyl group, a pyrrolyl group, a pyrazinyl group, a pyrimidinyl group, a pyridazinyl group, a selenazolyl group, a sulfolanyl group, a piperidinyl group, a pyrazolyl group, a tetrazolyl group, a morpholino group, etc.), a heteroaryloxy group (e.g., a 1-phenyltetrazol-5-oxy group, a 2-tetrahydropyranyloxy group, a pyridyloxy group, a thiazolyloxy group, an oxazolyloxy group, an imidazolyloxy group, etc.), an alkoxy group (e.g., a methoxy group, an ethoxy group, a propyloxy group, a tert-butoxy group, a pentyloxy group, a hexyloxy group, an octyloxy group, a dodecyloxy group, etc.), a cycloalkoxy group (e.g., a cyclopentyloxy group, a cyclohexyloxy group, etc.), an aryloxy group (e.g., a phenoxy group, a 2-naphthyloxy group, a 2-methylphenoxy group, a 4-tert-butylphenoxy group, a 3-nitrophenoxy group, etc.), an alkylthio group (e.g., a methylthio group, an ethylthio group, a propylthio group, a pentylthio group, a hexylthio group, an octylthio group, a dodecylthio group, etc.), a cycloalkylthio group (e.g., a cyclopentylthio group, a cyclohexylthio group, etc.), an arylthio group (e.g., a phenylthio group, a 1-naphthylthio group, etc.), a heteroarylthio group (e.g., a pyridylthio group, a thiazolylthio group, an oxazolylthio group, an imidazolylthio group, a furylthio group, a pyrrolylthio group, etc.), an alkoxycarbonyl group (e.g., a methoxycarbonyl group, an ethoxycarbonyl group, a butoxycarbonyl group, an octyloxycarbonyl group, a dodecyloxycarbonyl group, etc.), an aryloxycarbonyl group (e.g., a phenyloxycarbonyl group, a naphthyloxycarbonyl group, etc.), a sulfamoyl group (e.g., an aminosulfonyl group, a methylaminosulfonyl group, a dimethylaminosulfonyl group, a butylaminosulfonyl group, a hexylaminosulfonyl group, a cyclohexylaminosulfonyl group, an octylaminosulfonyl group, a dodecylaminosulfonyl group, a phenylaminosulfonyl group, a napthylaminosulfonyl group, a 2-pyridylaminosulfonyl group, a morpholinosulfonyl group, a pyrrolidinosulfonyl group, etc.), an ureido group (e.g., a methylureido group, an ethylureido group, a pentylureido group, a cyclohexylureido group, an octylureido group, a dodecylureido group, a phenylureido group, a naphthylureido group, a 2-pyridylaminoureido group, etc.), an acyl group (e.g., an acetyl group, an ethylcarbonyl group, a propylcarbonyl group, a pentylcarbonyl group, a cyclohexylcarbonyl group, an octylcarbonyl group, a 2-ethylhexylcarbonyl group, a dodecylcarbonyl group, a phenylcarbonyl group, a naphthylcarbonyl group, a pyridylcarbonyl group, etc.), an acyloxy group (e.g., a formyloxy group, an acetyloxy group, a pivaloyloxy group, a stearoyloxy group, a benzoyloxy group, a p-methoxyphenylcarbonyloxy group, an ethylcarbonyloxy group, a butylcarbonyloxy group, an octylcarbonyloxy group, a dodecylcarbonyloxy group, a phenylcarbonyloxy group, etc.), an acyloxycarbonyl group (e.g., an acetyloxycarbonyl group, a propionyloxycarbonyl group, etc.), an acylamino group (e.g., an acetylamino group, a benzoylamino group, a formylamino group, a pivaloylamino group, a lauroylamino group, etc.), a carbamoylamino group (e.g., an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethylhexylaminocarbonyl group, a dodecylaminocarbonyl group, a phenylaminocarbonyl group, a naphthylaminocarbonyl group, a 2-pyridylaminocarbonyl group, a morpholinocarbonyl group, a piperidinocarbonyl group, etc.), an alkanesulfinyl group or arylsulfinyl group (e.g., a methanesulfinyl group, an ethanesulfinyl group, a butanesulfinyl group, a cyclohexanesulfinyl group, a 2-ethylhexanesulfinyl group, a dodecanesulfinyl group, a phenylsulfinyl group, a naphthylsulfinyl group, a 2-pyridylsulfinyl group, etc.), an alkanesulfonyl or arylsulfonyl group (e.g., a methanesulfonyl group, an ethanesulfonyl group, a butanesulfonyl group, a cyclohexanesulfonyl group, a 2-ethylhexanesulfonyl group, a dodecanesulfonyl group, a phenylsulfonyl group, a naphthylsulfonyl group, a 2-pyridylsulfonyl group, etc.), an amino group (e.g., an amino group, a methylamino group, an ethylamino group, a dimethylamino group, a butylamino group, a cyclopentylamino group, a 2-ethylhexylamino group, a dodecylamino group, an anilino group, an N-methylanilino group, a diphenylamino group, a naphthylamino group, a 2-pyridylamino group, etc.), a silyloxy group (e.g., a trimethylsilyloxy group, a tert-butyldimethylsilyloxy group, etc.), an aminocarbonyloxy group (e.g., an N,N-dimethylcarbamoyloxy group, an N,N-diethylcarbamoyloxy group, a morpholinocarbonyloxy group, an N,N-di-n-octylaminocarbonyloxy group, an N-n-octylcarbamoyloxy group, etc.), an alkoxycarbonyloxy group (e.g., a methoxycarbonyloxy group, an ethoxycarbonyloxy group, a tert-butoxycarbonyloxy group, a n-octyloxycarbonyloxy group, etc.), an aryloxycarbonyloxy group (e.g., a phenoxycarbonyloxy group, a p-methoxyphenoxycarbonyloxy group, a p-n-hexadecyloxyphenoxycarbonyloxy group, etc.), an alkoxycarbonylamino group (e.g., a methoxycarbonylamino group, an ethoxycarbonylamino group, a tert-butoxycarbonylamino group, a n-octadecyloxycarbonylamino group, an N-methyl-methoxycarbonylamino group, etc.), an aryloxycarbonylamino group (e.g., a phenoxycarbonylamino group, a p-chlorophenoxycarbonylamino group, a m-n-octyloxyphenoxycarbonylamino group, etc.), a sulfamoylamino group (e.g., a sulfamoylamino group, an N,N-dimethylaminosulfonylamino group, an N-n-octylaminosulfonylamino group, etc.), a mercapto group, an arylazo group (e.g., a phenylazo group, a naphthylazo group, a p-chlorophenylazo group, etc.), a hetero aryl azo group (e.g., a pyridylazo group, a thiazolylazo group, an oxazolylazo group, an imidazolylazo group, a furylazo group, a pyrrolylazo group, a 5-ethylthio-1,3,4-thiadiazol-2-ylazo group, etc.), an imino group (e.g., an N-succinimid-1-yl group, an N-phthalimid-1-yl group, etc.), a phosphino group (e.g., a dimethylphosphino group, a diphenylphosphino group, a methylphenoxyphosphino group, etc.), a phosphinyl group (e.g., a phosphinyl group, a dioctyloxyphosphinyl group, a diethoxyphosphinyl group, etc.), a phosphinyloxy group (e.g., a diphenoxyphosphinyloxy group, a dioctyloxyphosphinyloxy group, etc.), a phosphinylamino group (e.g., a dimethoxyphosphinylamino group, a dimethylaminophosphinylamino group, etc.), a silyl group (e.g., a trimethylsiyl group, a tert-butyldimethylsilyl group, a phenyldimethylsilyl group, a trichlorosilyl group, etc.), an alkoxysilyl group (e.g., a trimethoxysilyl group, a triethoxysilyl group, etc.), a halocarbonyl group (e.g., a fluorocarbonyl group, a chlorocarbonyl group, a bromocarbonyl group, an iodocarbonyl group, etc.), an aldehydro group, a cyano group, a nitro group, a nitroso group, a hydroxyl group, a sulfo group, a carboxyl group, an azido group, an isocyanate group, and a thiol group. These substituents may further be substituted by other substituents, and the organic compounds may contain two or more of these substituents per molecule. In the case where the organic compound has two or more of these substituents per molecule, the substituents may be the same as or different from each other.

In the case where the organic compound is a fluorine-containing organic compound, fluorine atoms may be connected to the carbon atom or atoms of the parent hydrocarbon, to the carbon atom of the hydrocarbon of the substituent, or to other atom than carbon atom. It suffices for the fluorine-containing organic compound to have one or more fluorine atoms per molecule, and the compound may be perfluorinated.

The organic compound having a fluorine content of from 0 to 79% by weight may be a polymerizable compound. “Polymerizable compound” means a compound having at least one polymerizable functional group per molecule, and the functional group may contain a fluorine atom or atoms. Examples of the polymerizable group include an unsaturated double-bond functional group such as an acryloyl group, a methacryloyl group, a 2-fluoroacryloyl group, a 2-trifluoromethylacryloyl group, a 3,3-difluoroacryloyl group, an acrylamido group, a vinyl group, a trifluorovinyl group, an allyl group, a vinyl ether group or a perfluorovinyl ether group; an unsaturated triple-bond functional group such as an ethynyl group or a propargyl group; a sol-gel poly-condensatable group such as an alkoxysilyl group or a silanol group; a glycidyl group; and an oxetanyl group. The polymerizable compound may have two or more polymerizable functional groups per molecule and, in the case where the compound has two or more polymerizable functional groups per molecule, the functional groups may be the same as or different from each other. Also, the polymerizable compound may be in a single-component form or a multi-component form, and may be any of a monomer, a prepolymer (e.g., a dimer, a trimer, a tetramer, or an oligomer), and a mixture thereof.

The organic compound having a fluorine content of from 0 to 79% by weight may be a polymer. The polymer cannot particularly be specified, since it can properly be altered according to the use, but there can be illustrated polymers obtained by polymerizing the aforesaid polymerizable compounds.

The organic compound having a fluorine content of from 0 to 79% by weight cannot particularly be specified, since it can properly be altered according to the use, but the compound is preferably an organic compound having a solubility of 1% by weight or more in the perfluorochloroether solvent in the invention, more preferably an organic compound having a solubility of 10% by weight or more in the perfluorochloroether solvent, most preferably an organic compound having a solubility of 20% by weight or more in the perfluorochloroether solvent.

According to the invention, there can be obtained a liquid composition wherein both a fluorine-free organic compound and a fluorine-containing organic compound, or both an organic compound having a low fluorine content and an organic compound having a high fluorine content, are dissolved in a perfluorochloroether solvent containing a perfluorochloroether compound having a (chlorine content/fluorine content) value within the range of from 1.2 to 4.5.

For example, there can be obtained a liquid composition wherein an organic compound having a fluorine content of from 0 to 20% by weight is dissolved in a concentration of from 10 to 30% by weight and an organic compound having a fluorine content of from 30 to 79% by weight is dissolved in a concentration of from to 30% by weight, in a perfluorochloroether solvent containing a perfluorochloroether compound having a (chlorine content/fluorine content) value within the range of from 1.2 to 4.5.

(Use of the Perfluorochloroether-Containing Composition)

Next, uses of the perfluorochloroether-containing composition of the invention will be described below. However, the uses of the perfluorochloroether-containing composition of the invention are not limited only to them.

Since the perfluorochloroether solvent of the invention has extremely high dissolution capacity for both fluorine-free organic compounds and fluorine-containing organic compounds and is stable, the perfluorochloroether solvent-containing composition can find wide uses. Uses of the perfluorochloroether-containing composition of the invention are not particularly limited and, for example, the composition can be used as a coating agent, a detergent, a chemical reaction solution, a chemical reaction solvent, a cooling solvent, an extraction solvent, a solvent for chromatography, or the like. Effective components for other purposes may be dissolved or dispersed therein according to the use. Such effective components for other purposes may be inorganic compounds or organic compounds, and are not particularly limited, since they can be altered properly according to the use. For example, in the case of using the composition containing a polymerizable compound dissolved in the perfluorochloroether solvent as a coating agent, there can be dissolved or dispersed in the composition a polymerization initiator, a polymerization promoting agent, a chain transfer catalyst, a surfactant, a polymerization inhibitor, or the like. Also, in the case of using the composition in a chemical reaction solution, it can dissolve or disperse, for example, a metal catalyst, an inorganic salt, a mineral acid, or the like.

The perfluorochloroether solvent of the invention is extremely useful as a coating composition. In the case of preparing a coating composition wherein both a fluorine-free organic compound and a fluorine-containing organic compound are dissolved, use of a solvent having a low dissolution capacity for the fluorine-free organic compound or a solvent having a low dissolution capacity for the fluorine-containing organic compound fails to provide a uniform coating film, since the resulting coating solution does not become uniform. The perfluorochloroether solvent of the invention has such a high dissolution capacity for both a fluorine-free organic compound and a fluorine-containing organic compound that, even when a fluorine-free organic compound and an organic compound having a high fluorine content are used, the solvent can provide a uniform coating solution, thus being capable of providing a uniform coating film.

The content of the perfluorochloroether compound having a (chlorine content/fluorine content) value within the range of from 1.2 to 4.5 in the coating composition cannot be particularly limited, since it is properly changed depending upon solubility of the organic compound having a fluorine content of from 0 to 79% by weight, other additives, and upon intended use. For example, in the case of obtaining a composition wherein both a fluorine-free organic compound and a fluorine-containing organic compound are dissolved, the perfluorochloroether compound having a (chlorine content/fluorine content) value within the range of from 1.2 to 4.5 is contained in a content of preferably from 1 to 90% by weight, more preferably from 5 to 80% by weight in the coating composition in order to enhance solubility of both the fluorine-free organic compound and the fluorine-containing organic compound.

Since the perfluorochloroether solvent of the invention is inert to many reaction reagents, a composition containing the solvent is extremely useful as a chemical reaction solvent or as a chemical reaction solution. Use of the perfluorochloroether solvent-containing composition of the invention as a chemical reaction solvent or as a chemical reaction solution is not particularly limited as long as the reaction condition is not a condition under which the perfluorochloroether solvent reacts with a reaction substrate or a reaction reagent to decompose and, for example, it can favorably be used in general organic chemical reactions such as oxidation reaction, reduction reaction, addition reaction, substitution reaction, cycloaddition reaction, esterification reaction, amidation reaction, hydrolysis reaction, radical reaction, halogenation reaction, reaction using an organometallic reagent such as Grignard reagent, and polymerization reaction. The solvent can particularly favorably be used as a solvent or as a reaction solution for liquid-phase fluorination.

The content of the perfluorochloroether compound having a (chlorine content/fluorine content) value within the range of from 1.2 to 4.5 in the chemical reaction solvent or chemical reaction solution cannot be particularly limited, since it is properly changed depending upon solubility of the organic compound having a fluorine content of from 0 to 79% by weight, chemical reaction to be employed, and upon reactive agents. For example, in the case of using for the liquid phase fluorination reaction to be described hereinafter, the perfluorochloroether compound having a (chlorine content/fluorine content) value within the range of from 1.2 to 4.5 is contained in a content of preferably from 1 to 99.9% by weight, more preferably from 5 to 99% by weight in the reaction solution in order to enhance solubility of both the fluorine-free organic compound and the fluorine-containing organic compound.

The liquid-phase fluorination reaction is a reaction similar to that described in U.S. Pat. No. 5,093,432. That is, the liquid-phase fluorination reaction is a reaction of partially substituting hydrogen atoms with fluorine atoms or substituting all hydrogen atoms with fluorine atoms (perfluorination) by reacting a hydrogen-containing organic compound with a fluorine gas in a solvent which is inert to the fluorine gas, such as a perfluoro solvent. Also, a reaction of adding fluorine to an unsaturated bond of an organic compound having an unsaturated bond such as a double bond is included in the liquid-phase fluorination reaction. In the liquid-phase fluorination reaction, solubility of a reaction substrate to be fluorinated and solubility of a product of fluorinated compound in the solvent is extremely important with respect to yield and purity of the product. That is, in the case of fluorinating or perfluorinating a fluorine-free organic compound by the liquid-phase fluorination reaction, a fluorine-free organic compound having a low solubility in a perfluoro solvent is scarcely soluble in the solvent and, therefore, the fluorine-free organic compound and the solvent form separate two layers. Therefore, side reactions proceed, and the yield and purity of the product are liable to be reduced, thus such solvent not being preferred. Also, in the case where the solubility of a fluorine-containing organic compound in a perfluoro solvent is low, the fluorinated compound is scarcely soluble in the solvent. Therefore, side reactions proceed, and the yield and purity of the product are liable to be reduced, thus such solvent not being preferred. Accordingly, a solvent to be used for the liquid-phase reaction solvent is required to have high dissolution capacity for both a fluorine-free organic compound and a fluorine-containing organic compound.

As has been described hereinbefore, the perfluorochloroether solvent in the invention has extremely high dissolution capacity for both a fluorine-containing organic compound and a fluorine-free organic compound and is, therefore, extremely useful as a solvent for liquid-phase fluorination reaction and can favorably be used as a solvent for liquid-phase fluorination reaction of a fluorine-free organic compound and a fluorine-containing organic compound.

In the case of using the perfluorochloroether solvent in the invention as a solvent for the liquid-phase fluorination reaction, the solvent may be used alone or as a mixture with other perfluoro solvent. Examples of such perfluoro solvent include perfluoroalkane compounds [e.g., FC-72 (trade name; manufactured by Sumitomo 3M), etc.], perfluoroether compounds [FC-75, FC-77 (both being trade names; manufactured by Sumitomo 3M), etc.], perfluoropolyether compounds [trade name: Krytox® (trademark of DuPont); trade name: Fomblin® (trademark of Solvay Solexis); trade name: Galden® (trademark of Solvay Solexis); trade name: Demnum; manufactured by Daikin Industries; etc.], chlorofluorocarbon compounds [CFC-11, CFC-113, etc.], and inert fluids [trade name: Fluorrinert® (trade name of 3M); trade name: Halocarbon (trademark of Halocarbon Co.); etc.]. These may be used as a mixture of two or more thereof.

In the case of using the perfluorochloroether solvent of the invention which contains a perfluorochloroether compound having the (chlorine content/fluorine content) value in the range of from 1.2 to 4.5 as a solvent for the liquid-phase fluorination reaction solvent, the solvent can preferably dissolve the starting material of fluorine-free organic compound or fluorine-containing organic compound and the product of perfluorinated compound in contents of 20% by weight or more, respectively. It is particularly preferred for the solvent to dissolve the starting material of the liquid-phase fluorination reaction of a fluorine-free organic compound or fluorine-containing organic compound and the product of perfluorinated compound in about the same amounts, because such solvent permits the fluorination reaction to proceed effectively.

### EXAMPLES

Examples specifically illustrating the present invention will be described below, but the invention is not restricted at all by them. Here, nuclear magnetic resonance method is abbreviated as NMR, gas chromatography as GC, and gas chromatography mass spectrometry as GC-MS. In ¹H-NMR, measurement is conducted using tetramethylsilane (TMS) as an internal standard. In ¹⁹F-NMR, measurement is conducted using fluorotrichloromethane as an external standard.

### Example 1

### Preparation of Compound (2)

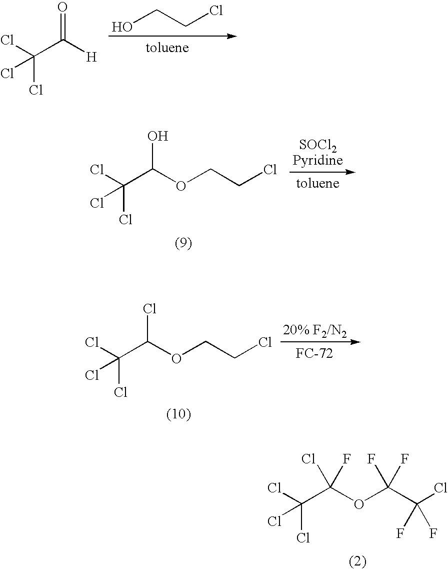

(1-1) Preparation of Compound (10)

30 g (373 mmol) of 2-chloroethanol is placed in a glass-made reaction vessel, and 30 ml of toluene is added thereto, followed by cooling to 10° C. or lower than that. 109.8 g (745 mmol) of chloral is added thereto, and the mixture is stirred at room temperature for 2 hours to obtain compound (9). The reaction solution is cooled to 10° C. or lower than that, and 66.6 g (560 mmol) of thionyl chloride and 44.3 g (560 mmol) of pyridine are added thereto, followed by stirring at room temperature for 3 hours. After cooling the reaction solution to 10° C. or lower than that, 100 mL of water is added thereto, followed by extracting with 50 mL of toluene. The organic layer is washed with 100 mL of a 15% aqueous solution of sodium chloride and 100 mL of a 7.5% aqueous solution of sodium bicarbonate (twice), then with 100 mL of an aqueous solution saturated with sodium chloride, and is dehydrated over anhydrous sodium sulfate to dry. After filtration, the solvent is distilled off under reduced pressure to obtain 49.4 g (201 mmol; yield: 53.8%) of compound (10). The thus obtained compound (10) has a purity of 96% measured by GC.

Compound (10): ¹H-NMR [CDCl₃]: δ[ppm]=3.75 (2H, dd, J=6.0 Hz, 4.8 Hz), 3.94-4.02 (1H, m), 4.22-4.30 (1H, m), 5.82 (1H, S); GC-MS [SCI]: m/z=209 [M⁺-Cl]

(1-2) Preparation of Compound (2)

300 mL of FC-72 (trade name; manufactured by Sumitomo 3M) and 63.6 g (1.51 mol) of sodium fluoride are placed in a 500-mL, Teflon (registered trademark)-made reaction vessel, and the external temperature is kept at about −25° C. At the outlet of the reaction vessel are provided in series a NaF pellet-filled layer and a cooling device kept at −40° C., and a liquid formed by condensation in the cooling device is allowed to return to the reaction vessel via a return line. After introducing into the reactor a helium gas at a rate of 200 mL/min for 30 minutes, a fluorine gas diluted to 20% with a nitrogen gas (hereinafter merely referred to as “fluorine gas”) is introduced at a rate of 250 mL/min for 15 minutes. While introducing the fluorine gas at the same rate, a mixture of 20 g (81.2 mmol) of compound (10), 20 g (98.6 mmol) of AK-225 (trade name; manufactured by AGC), and 20 g (107.5 mmol) of hexafluorobenzene are added thereto over 8.7 hours. Thereafter, while introducing thereinto the fluorine gas at the same rate, a mixture of 1 g (5.37 mmol) of hexafluorobenzene and 1 g of FC-72 is added thereto over 30 minutes. After introducing a helium gas at a rate of 200 mL/min for 1 hour, the reaction solution is filtered, and FC-72 is distilled off under ordinary pressure. Distillation of the concentrate under reduced pressure gives 15.9 g (47.3 mmol; yield: 58.2%) of compound (2). The thus obtained compound (2) has a purity of 91% measured by GC.

Compound (2): ¹⁹F-NMR [CDCl₃]: δ[ppm]=−89.2 (1F), −86.9 (1F), −73.7 (2F), −71.1 (1F); GC-MS [SCI]: m/z=299 [M⁺-Cl]

### Example 2

### Preparation of Compound (3)

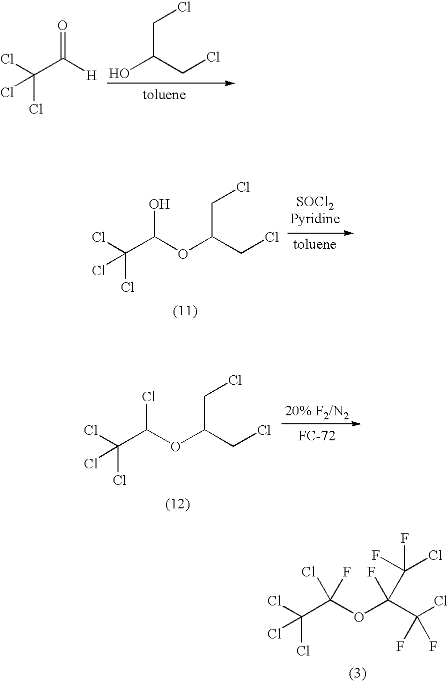

(2-1) Preparation of Compound (12)

10 g (77.5 mmol) of 1,3-dichloro-2-propanol is placed in a glass-made reaction vessel, and 10 ml of toluene is added thereto, followed by cooling to 10° C. or lower than that. 45.6 g (310 mmol) of chloral is added thereto, and the mixture is stirred at 60° C. for 3 hours to obtain compound (11). The reaction solution is cooled to 10° C. or lower than that, and 18.5 g (156 mmol) of thionyl chloride and 12.3 g (155 mmol) of pyridine are added thereto, followed by stirring at room temperature for 3 hours. After cooling the reaction solution to 10° C. or lower than that, 30 mL of water is added thereto, followed by extracting with 10 mL of toluene. The organic layer is washed with 30 mL of a 15% aqueous solution of sodium chloride and 30 mL of a 7.5% aqueous solution of sodium bicarbonate (twice), then with 30 mL of an aqueous solution saturated with sodium chloride, and dehydrated over anhydrous sodium sulfate to dry. After filtration, the solvent is distilled off under reduced pressure to obtain 15.5 g (52.6 mmol; yield: 67.8%) of compound (12). The thus obtained compound (12) has a purity of 95% measured by GC.

Compound (12): ¹H-NMR [CDCl₃]: δ[ppm]=3.70-3.90 (4H, m), 4.23-4.35 (1H, m), 5.99 (1H, S); GC-MS [SCI]: m/z=257 [M⁺-Cl]

(2-2) Preparation of Compound (3)

The same apparatus as in Example 1 is set up, and 300 mL of FC-72 and 18.5 g (441 mmol) of sodium fluoride are placed in a 500-mL, Teflon-made reaction vessel, and then the external temperature is kept at about −25° C. After introducing into the reaction vessel a helium gas at a rate of 200 mL/min for 30 minutes, a fluorine gas is introduced at a rate of 200 mL/min for 15 minutes. While introducing the fluorine gas at the same rate, a mixture of 5.5 g (18.7 mmol) of compound (12), 5.5 g (27.1 mmol) of AK-225 (trade name; manufactured by AGC), and 5.5 g (29.6 mmol) of hexafluorobenzene are added thereto over 3 hours. Thereafter, while introducing the fluorine gas at the same rate, a mixture of 2 g (10.7 mmol) of hexafluorobenzene and 2 g of FC-72 is added thereto over 30 minutes. After introducing a helium gas at a rate of 200 mL/min for 1 hour, the reaction solution is filtered, and FC-72 is distilled off under ordinary pressure. Distillation of the consentrate under reduced pressure gives 5.66 g (14.1 mmol; yield: 75.1%) of compound (3). The thus obtained compound (3) has a purity of 96% measured by GC.

Compound (3): ¹⁹F-NMR [CDCl₃]: δ[ppm]=−137.1 (1F), −63.1-−65.2 (5F); GC-MS [SCI, 70 eV]: m/z=365 [M⁺-Cl]

### Example 3

### Preparation of Compound (4)

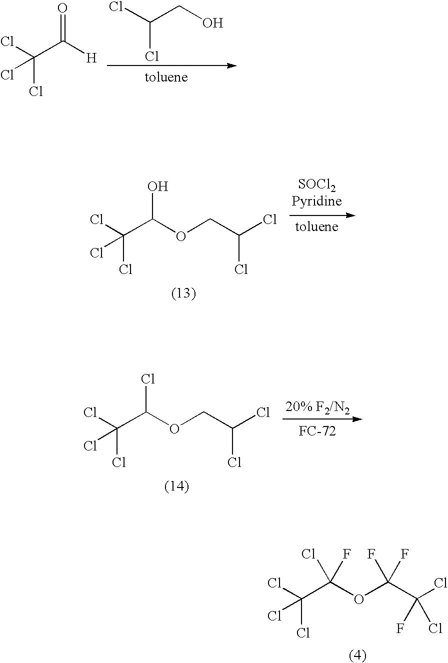

(3-1) Preparation of Compound (14)

15 g (130 mmol) of 2,2-dichloroethanol is placed in a glass-made reaction vessel, and 15 ml of toluene is added thereto, followed by cooling to 10° C. or lower than that. 76.9 g (522 mmol) of chloral is added thereto, and the mixture is stirred at 30° C. for 3 hours to obtain compound (13). The reaction solution is cooled to 10° C. or lower than that, and 30.9 g (260 mmol) of thionyl chloride and 20.6 g (260 mmol) of pyridine are added thereto, followed by stirring at room temperature for 3 hours. After cooling the reaction solution to 10° C. or lower than that, 45 mL of water is added thereto, followed by extracting with 15 mL of toluene. The organic layer is washed with 45 mL of a 15% aqueous solution of sodium chloride and 45 mL of a 7.5% aqueous solution of sodium bicarbonate (twice), then with 45 mL of an aqueous solution saturated with sodium chloride, and dehydrated over anhydrous sodium sulfate to dry. After filtration, the solvent is distilled off under reduced pressure to obtain 26.1 g (93.0 mmol; yield: 71.5%) of compound (14). The thus obtained compound (14) has a purity of 94% measured by GC.

Compound (14): ¹H-NMR [CDCl₃]: δ[ppm]=4.30 (1H, dd, J=4.8, 4.5 Hz), 4.42 (1H, dd, J=4.8, 4.6), 5.65 (1H, dd, J=4.6, 4.5 Hz), 6.09 (1H, S); GC-MS [SCI]: m/z=243 [M⁺-Cl]

(3-2) Preparation of Compound (4)

The same apparatus as in Example 1 is set up, and 300 mL of FC-72 and 24.1 g (575 mmol) of sodium fluoride are placed in a 500-mL, Teflon-made reaction vessel, and then the external temperature is kept at about −25° C. After introducing into the reaction vessel a helium gas at a rate of 200 mL/min for 30 minutes, a fluorine gas is introduced at a rate of 200 mL/min for 15 minutes. While introducing the fluorine gas at the same rate, a mixture of 10 g (35.6 mmol) of compound (14), 10 g (49.3 mmol) of AK-225 (trade name; manufactured by AGC), and 10 g (53.7 mmol) of hexafluorobenzene are added thereto over 4.6 hours. Thereafter, while introducing the fluorine gas at the same rate, a mixture of 2 g (10.7 mmol) of hexafluorobenzene and 2 g of FC-72 is added thereto over 30 minutes. After introducing a helium gas at a rate of 200 mL/min for 1 hour, the reaction solution is filtered, and FC-72 is distilled off under ordinary pressure. Distillation of the consentrate under reduced pressure gives 9.39 g (26.6 mmol; yield: 74.8%) of compound (4). The thus obtained compound (4) has a purity of 98% measured by GC.

Compound (4): ¹⁹F-NMR [CDCl₃]: δ[ppm]=−87.8 (2F), −76.2 (1F), −71.9 (1F); GC-MS [SCI]: m/z=315 [M⁺-Cl]

### Example 4

### Preparation of Compound (5)

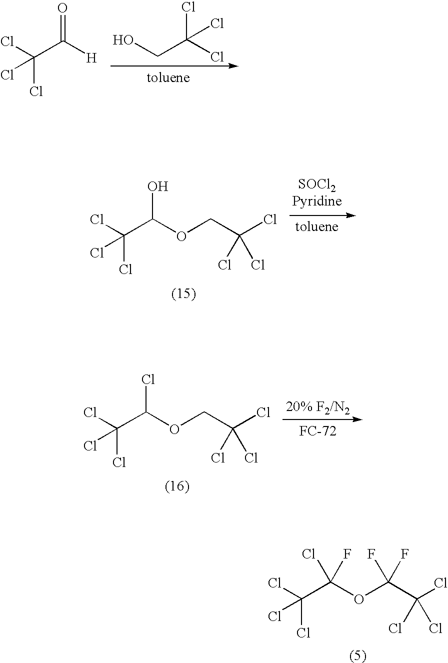

(4-1) Preparation of Compound (16)

10 g (66.9 mmol) of 2,2,2-trichloroethanol is placed in a glass-made reaction vessel, and 10 ml of toluene is added thereto, followed by cooling to 10° C. or lower than that. 98.6 g (669 mmol) of chloral is added thereto, and the mixture is stirred at 60° C. for 3 hours to obtain compound (15). The reaction solution is cooled to 10° C. or lower than that, and 15.9 g (134 mmol) of thionyl chloride and 10.6 g (134 mmol) of pyridine are added thereto, followed by stirring at room temperature for 3 hours. After cooling the reaction solution to 10° C. or lower than that, 30 mL of water is added thereto, followed by extracting with 10 mL of toluene. The organic layer is washed with 30 mL of a 15% aqueous solution of sodium chloride and 30 mL of a 7.5% aqueous solution of sodium bicarbonate (twice), then with 30 mL of an aqueous solution saturated with sodium chloride, and dehydrated over anhydrous sodium sulfate to dry. After filtration, the solvent is distilled off under reduced pressure to obtain 10.8 g (34.3 mmol; yield: 51.3%) of compound (16). ¹H-NMR spectrum of the thus obtained compound (16) coincides with the spectrum of the standard sample described in Justus Liebigs Annalen der Chemie, vol. 755, pp. 40-50 (1972). The thus obtained compound (16) has a purity of 94% measured by GC.

(4-2) Preparation of Compound (5)

The same apparatus as in Example 1 is set up, and 300 mL of FC-72 and 18.2 g (433 mmol) of sodium fluoride are placed in a 500-mL, Teflon-made reaction vessel, and then the external temperature is kept at about −25° C. After introducing into the reaction vessel a helium gas at a rate of 200 mL/min for 30 minutes, a fluorine gas is introduced at a rate of 200 mL/min for 15 minutes. While introducing the fluorine gas at the same rate, a mixture of 10 g (31.7 mmol) of compound (16), 10 g (49.3 mmol) of AK-225 (trade name; manufactured by AGC), and 10 g (53.7 mmol) of hexafluorobenzene are added thereto at a rate of 4.5 mL/h. Thereafter, while introducing the fluorine gas at the same rate, a mixture of 2 g (10.7 mmol) of hexafluorobenzene and 2 g of FC-72 is added thereto over 30 minutes. After introducing a helium gas at a rate of 200 mL/min for 1 hour, the reaction solution is filtered, and FC-72 is distilled off under ordinary pressure. Distillation of the consentrate under reduced pressure gives 9.66 g (26.2 mmol; yield: 82.5%) of compound (5). The thus obtained compound (5) has a purity of 98% measured by GC.

Compound (5): ¹⁹F-NMR [CDCl₃]: δ[ppm]=−88.9 (2F), −72.6 (1F); GC-MS [SCI]: m/z=331 [M⁺-Cl]

### Example 5

### Measurement of Solubility

The following compound (17) is gradually added to 1 g of the compound (2) under stirring. Even when 2 g of the compound (17) is added, the resulting solution remains uniform, and thus it is found that the compound (17) is completely soluble in the compound (2). Next, when the compound (2) is gradually added to 1 g of the compound (17) under stirring, the resulting solution remains uniform even when 2 g of the compound (2) is added. Thus, it is found that the compound (17) is completely soluble in the compound (2). It is found from the above results that the compound (17) is soluble in the compound (2) in any proportion.

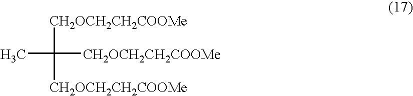

### Example 6

### Measurement of Solubility

The same procedures as in Example 5 are conducted except for changing the compound (2) to the compound (3), and it is found that the compound (17) is soluble in the compound (3) in any proportion.

### Example 7

### Measurement of Solubility

The same procedures as in Example 5 are conducted except for changing the compound (2) to the compound (4), and it is found that the compound (17) is soluble in the compound (4) in any proportion.

### Example 8

### Measurement of Solubility

The same procedures as in Example 5 are conducted except for changing the compound (2) to the compound (5), and it is found that the compound (17) is soluble in the compound (5) in any proportion.

### Comparative Example 1

### Measurement of Solubility

The compound (17) is gradually added to 3 g of Halocarbon 1.8 oil [manufactured by Halocarbon Co.]. When 2 g of the compound (17) is added, the amount exceeds the saturation solubility of the compound (17) in Halocarbon 1.8 oil, and hence the solution separates into two layers, one being a layer of Halocarbon 1.8 (lower layer) and the other being a layer of the compound (17) (upper layer). 1.67 g of the lower Halocarbon 1.8 oil layer saturated with the compound (17) is withdrawn, and 150 μL (0.867 mmol) of 1,1,2,2-tetrachloroethane (manufactured by Wako Pure Chemical Industries, Ltd.) is added thereto as an internal standard. The resulting solution is diluted with deutrochloroform, followed by measuring ¹H-NMR. The amount of the compound (17) in 1.67 g of the Halocarbon 1.8 oil layer is determined from integral ratio of 1,1,2,2-tetrachloroethane to the compound (17). As a result, it is found that the amount of the compound (17) is 0.27 g, and that the saturated solubility of the compound (17) in Halocarbon 1.8 oil is 16% by weight.

### Comparative Example 2

### Measurement of Solubility

The same procedures as in Comparative Example 1 are conducted except for changing Halocarbon 1.8 oil to the following compound (18) described in JP-T-4-500520. The saturated solubility of the compound (17) in the compound (18) is found to be 4.7% by weight.

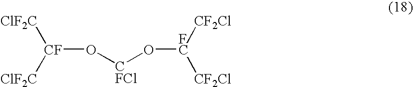

### Comparative Example 3

### Measurement of Solubility

The same procedures as in Comparative Example 1 are conducted except for changing Halocarbon 1.8 oil to the following FC-72 (perfluorohexane). The saturated solubility of the compound (17) in FC-72 is found to be 0.14% by weight.

CF₃(CF₂)₄CF₃

FC-72

Also, it can be confirmed that the compound (2), compound (3), compound (4), compound (5), Halocarbon 1.8 oil, and compound (18) used in Examples 5 to 8, and Comparative Examples 1 and 2, respectively, can dissolve FC-72 in any proportion.

Results of measurement of solubility conducted in Examples 5 to 8 and in Comparative Examples 1 to 3 can be tabulated in the following Table 1. In Table 1, boiling points and (chlorine content/fluorine content) values of the fluorine-containing solvents are also given.

TABLE 1

| | | | Solubility (% by weight) | |
|---|---|---|---|---|
| | Boiling | {(Chlorine Content)/ | Compound (17) | FC-72 (Fluorine |
| | Point/° C. | (Fluorine Content)} | (Fluorine Content: | Content: 79% by |
| Solvent | 760 mmHg | Value | 0% by weight) | weight) |
| Example 5 | 146 | 1.87 | soluble in any | soluble in any |
| Compound (2) | | | proportion | proportion |
| Example 6 | 200 | 1.87 | soluble in any | soluble in any |
| Compound (3) | | | proportion | proportion |
| Example 7 | 187 | 2.79 | soluble in any | soluble in any |
| Compound (4) | | | proportion | proportion |
| Example 8 | 221 | 4.35 | soluble in any | soluble in any |
| Compound (5) | | | proportion | proportion |
| Comparative | 205 | 1.14 | 16 | soluble in any |
| Example 1 | | | | proportion |
| Halocarbon 1.8 oil | | | | |
| Comparative | 202 | 0.718 | 4.7 | soluble in any |
| Example 2 | | | | proportion |
| Compound (18) | | | | |
| Comparative | 56 | 0 | 0.14 | — |
| Example 3 FC-72 | | | | |

As is shown in Table 1, it is found that the solubility of compound (17) increases with the increase of the (chlorine content/fluorine content) value, and that the perfluoro solvents having the (chlorine content/fluorine content) value within a range of the invention dissolve both the compound (17) and FC-72 in any proportion.

### Example 9

### Preparation of Compound (2) by Liquid-Phase Fluorination of Compound (10) Using Compound (2) as a Solvent

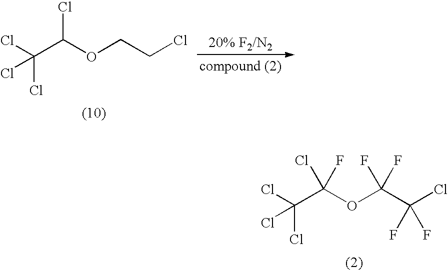

The same apparatus as in Example 1 is set up, and 100 mL of the compound (2) and 8.52 g (203 mmol) of sodium fluoride are placed in a 300-mL, Teflon-made reaction vessel, and then the mixture is kept at −10° C. or lower than that. After introducing into the reaction vessel a helium gas at a rate of 100 mL/min for 30 minutes, a fluorine gas is introduced at a rate of 100 mL/min for 15 minutes. While introducing the fluorine gas at the same rate, a mixture of 5 g (20.3 mmol) of the compound (10) and 5 g of the compound (2) are added thereto over 2.7 hours. Thereafter, while introducing the fluorine gas at the same rate, a mixture of 0.1 g (0.537 mmol) of hexafluorobenzene and 0.1 g of the compound (2) is added thereto over 15 minutes. After introducing a helium gas at a rate of 100 mL/min for 1 hour, the reaction solution is filtered. Analysis by GC and NMR reveals that all of the compound (10) is converted to the compound (2).

### Example 10

### Preparation of Compound (20) by Fluorination of Compound (17) Using Compound (2) as a Solvent

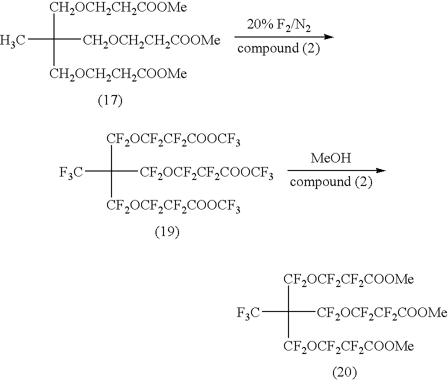

The same apparatus as in Example 1 is set up, and 100 mL of the compound (2) and 6.66 g (159 mmol) of sodium fluoride are placed in a 300-mL, Teflon-made reaction vessel, and then the mixture is kept at −10° C. or lower than that. After introducing into the reaction vessel a helium gas at a rate of 100 mL/min for 30 minutes, a fluorine gas is introduced at a rate of 100 mL/min for 15 minutes. While introducing the fluorine gas at the same rate, a mixture of 1 g (2.64 mmol) of the compound (17) and 1 g of the compound (2) are added thereto over 2.1 hours. Thereafter, while introducing the fluorine gas at the same rate, a mixture of 0.25 g (1.34 mmol) of hexafluorobenzene and 0.25 g of the compound (2) is added thereto over 30 minutes. After introducing a helium gas at a rate of 100 mL/min for 1 hour, the reaction solution is filtered to obtain a solution of the compound (19) in the compound (2). 0.665 g (15.8 mmol) of sodium fluoride and 64 mL of methanol are added thereto and, after stirring at room temperature for 1 hour, the solvent is distilled off under ordinary pressure to obtain 1.96 g (2.59 mmol; yield: 98.1%) of the compound (20). Analysis by GC reveals that purity of the compound (20) is 86%.

### Example 11

### Preparation of Compound (20) by Fluorination of Compound (17) Using Compound (5) as a Solvent

Preparation of the compound (20) is conducted in the same manner as in Example 10 except for changing the solvent from the compound (2) to the compound (5). The amount of the obtained compound (20) is 1.96 g (2.59 mmol; yield: 98.1%), and the GC purity thereof is 85%.

### Comparative Example 4

### Preparation of Compound (20) by Fluorination of Compound (17) Using FC-72 as a Solvent

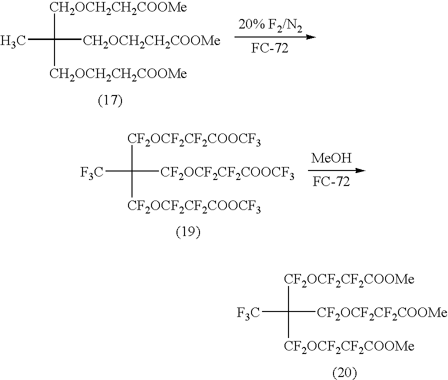

The same apparatus as in Example 1 is set up, and 100 mL of FC-72 and 6.66 g (159 mmol) of sodium fluoride are placed in a 300-mL, Teflon-made reaction vessel, and then the mixture is kept at −10° C. or lower than that. After introducing into the reaction vessel a helium gas at a rate of 100 mL/min for 30 minutes, a fluorine gas is introduced at a rate of 100 mL/min for 15 minutes. While introducing the fluorine gas at the same rate, a mixture of 1 g (2.64 mmol) of the compound (17) and 3 g (14.8 mmol) of AK-225 are added thereto over 2.5 hours. Thereafter, while introducing the fluorine gas at the same rate, a mixture of 2 g (10.7 mmol) of hexafluorobenzene and 2 g of FC-72 is added thereto over 1 hour. After introducing a helium gas at a rate of 100 mL/min for 1 hour, the reaction solution is filtered to obtain a solution of the compound (19) in FC-72. 0.665 g (15.8 mmol) of sodium fluoride and 64 mL of methanol are added thereto and, after stirring at room temperature for 1 hour, the solvent is distilled off under ordinary pressure to obtain 1.89 g (2.50 mmol; yield: 94.5%) of the compound (20). Analysis by GC reveals that purity of the compound (20) is 41%.

In Example 9, the compound (2) having a fluorine content of 28% by weight is obtained by perfluorinating the compound (10) having a fluorine content of 0% by weight. The solution is uniform throughout the period of from initiation to the completion of the reaction, with the compound (10) and intermediates wherein hydrogen atoms of the compound (10) are partially substituted by fluorine atoms being completely dissolved in the solvent (2). Also, since the compound (2) formed by perfluorination of the compound (10) is used as the solvent for the reaction, only the compound (2) exists after completion of the reaction, and hence purification is not necessary, thus the process being extremely useful for preparing the compound (2).

In Examples 10 and 11, the compound (17) having a fluorine content of 0% by weight is perfluorinated to obtain the compound (19) having a fluorine content of 62% by weight, and the compound (20) having a fluorine content of 53% by weight is obtained from the compound (19). In the fluorination reaction of the compound (17) to the compound (19), the solution is uniform throughout the reaction, with the compound (17), intermediates wherein hydrogen atoms of the compound (17) are partially substituted by fluorine atom, and the compound (19) being completely dissolved in the compound (2) or in the compound (5).

In Comparative Example 4, the reaction solution is not uniform, since the compound (17) is not soluble in FC-72. The product (20) in Comparative Example 4 has a purity of 41%, whereas the products (20) in Examples 10 and 11 have a purity of 86% and 85%, respectively. As is apparent from this, it is revealed that use of the perfluorochloroether solvent of the invention enhances solubility of both the fluorine-free organic compound and the fluorine-containing organic compound, which serves to suppress side-reactions and allow the fluorination reaction to proceed extremely smoothly.

### Example 12

### Preparation of Coating Film Using a Composition Containing a Fluorine-Containing Acrylate

40 parts by weight of 1,6-bis(acryloyloxy)-2,2,3,3,4,4,5,5-octafluorohexane [manufactured by Tokyo Chemical Industry Co., Ltd.; fluorine content: 41%], 4 parts by weight of a photo initiator of Irgacure 184 (manufactured by Ciba-Geigy Ltd.; fluorine content: 0% by weight), and 60 parts by weight of a solvent of the compound (2) are mixed to prepare a uniform curable coating solution. This coating solution is spin-coated on a 100-μm thick PET film. Then, curing is conducted by irradiating 400-mJ/cm² UV rays using a UV ray-irradiating apparatus having a 120-W high-pressure mercury lamp to thereby prepare a PET film having a uniform hard coat film formed on the surface thereof.

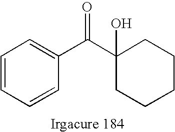

### Example 13

### Preparation of Coating Film Using a Composition Containing a Fluorine-Containing Acrylate and a Fluorine-Free Acrylate

120 parts by weight of dipentaerythritol hexaacrylate [manufactured by Nippon Kayaku; fluorine content: 0% by weight], 20 parts by weight of 1,6-bis(acryloyloxy)-2,2,3,3,4,4,5,5-octafluorohexane [manufactured by Tokyo Chemical Industry Co., Ltd.; fluorine content: 41%], 4 parts by weight of a photo initiator of Irgacure 184 (manufactured by Ciba-Geigy Ltd.; fluorine content: 0% by weight), and 60 parts by weight of a solvent of the compound (4) are mixed to prepare a uniform curable composition. This composition is spin-coated on a 100-μm thick PET film. Then, curing is conducted by irradiating 400-mJ/cm² UV rays using a UV ray-irradiating apparatus having a 120-W high-pressure mercury lamp to thereby prepare a PET film having a uniform hard coat film formed on the surface thereof.

### Example 14

### Preparation of Coating Film Using a Composition Containing a Siloxane Polymer

0.1 g of 3,3,3-trifluoropropyltrimethoxysilane [manufactured by Wako Pure Chemical Industries, Ltd.; fluorine content: 28%], 2 g of the compound (2), and 0.5 g of isopropyl alcohol (fluorine content: 0% by weight) are mixed and well stirred. 1.0 g of 1% HCl aqueous solution is gradually dropwise added to the mixture. After completion of the dropwise addition, the mixture is kept at 25° C. and allowed to stand for 7 days to prepare a coating composition. This composition is spin-coated on a 30-mm square glass piece. The coated glass piece is placed in a 100° C. thermostatic chamber and kept there for 12 hours to thereby prepare a uniform coating film.

### Example 15

### Preparation of a Coating Film Using a Composition Containing a Urethane Group-Containing Siloxane Polymer

(15-1) Preparation of a Coating Composition

1.07 g (4.33 mmol) of 3-(triethoxysilyl)propyl isocyanate [manufactured by Tokyo Chemical Industry Co., Ltd.] is added to a mixture of 1.0 g (2.16 mmol) of 1H,1H,10H,10H-hexadecafluoro-1,10-decanediol [manufactured by Tokyo Chemical Industry Co., Ltd.], 0.69 g (5 mmol) of potassium carbonate, and 5 mL of the compound (3), followed by stirring the resulting mixture at room temperature for 4 hours. After filtering through celite, 20 mL of the compound (3) is added thereto to obtain solution A. 0.88 g (8.8 mmol) of acetylacetone, 1.0 g (4.4 mmol) of tetraethyl orthotitanate [manufactured by Tokyo Chemical Industry Co., Ltd.], and 18.9 g of ethanol are mixed and, after mixing at room temperature for 10 minutes, 0.15 g (8.3 mmol) of water is added thereto, followed by stirring the resulting mixture at room temperature for 1 hour to obtain solution B. 0.3 g of water is added to a mixture of 2.5 mL of the solution A, 1.01 g of the solution B, and 2.5 mL of the compound (3), and the resulting mixture is stirred at room temperature for 4 hours, then allowed to stand for 14 hours to obtain a coating composition.

(15-2) Preparation of a Coating Film

The coating composition is spin-coated on a 30-mm square glass piece. The thus-coated glass piece is placed in a 150° C. thermostatic chamber and kept there for 30 minutes to thereby prepare a uniform coating film.

### Comparative Example 5

### Preparation of a Coating Film Using a Composition Containing FC-72

The same procedures as in Example 13 are conducted except for changing the compound (4) to FC-72. Dipentaerythritol hexaacrylate is insoluble in FC-72, and hence a uniform hard coat film is not obtained.

### Comparative Example 6

### Preparation of a Coating Film Using a Composition Containing FC-72

The same procedures as in Example 15 are conducted except for changing the compound (3) to FC-72. 3-(triethoxysilyl)propyl isocyanate, 1H,1H,10H,10H-hexadecafluoro-1,10-decanediol, and the like are not completely soluble in FC-72, and the solution A and the solution B are not uniformly mixed with each other, and hence a uniform hard coat film is not obtained.

### Example 16

### Preparation of Compound (2′)

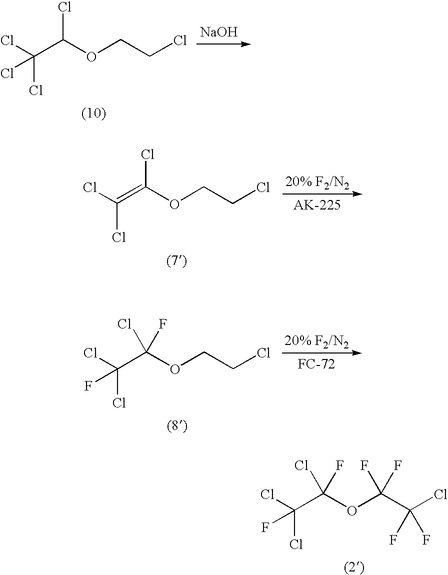

(1′-1) Preparation of Compound (7′)

5.84 g (23.7 mmol) of compound (10) and 112 mL of ethanol are placed in a glass-made reaction vessel, followed by cooling the mixture to an internal temperature of 10° C. or lower. 20 mL of 7.1% by weight sodium hydroxide aqueous solution is added thereto, and the resulting mixture is stirred at room temperature for 3 hours. After neutralizing the reaction solution with 1N hydrochloric acid, 150 mL of ethyl acetate and 100 mL of 10% saline solution are added thereto, followed by liquid separation. The organic layer is washed with 25% saline solution, and dried over sodium sulfate. After filtration, the solvent is distilled off under reduced pressure to obtain 4.51 g (21.5 mmol; yield: 90.7%) of compound (7′).

(1′-2) Preparation of Compound (8′)

The same apparatus as in Example 1 is set up, and 4 g (19.1 mmol) of compound (7′) and 100 mL of AK-225 are placed in a 300-mL, Teflon-made reaction vessel, and then the external temperature is kept at about −98° C. After introducing into the reaction vessel a helium gas at a rate of 100 mL/min for 30 minutes, a fluorine gas is introduced at a rate of 20 mL/min for 160 minutes. After introducing a helium gas at a rate of 100 mL/min for 30 minutes, the reaction solution is concentrated under reduced pressure to obtain 4.26 g (17.2 mmol; yield: 90.1%) of compound (8′).

(1′-3) Preparation of Compound (2′)

The same apparatus as in Example 1 is set up, and 7.05 g (168 mmol) of sodium fluoride and 100 mL of FC-72 are placed in a 500-mL, Teflon-made reaction vessel, and then the external temperature is kept at about −25° C. After introducing into the reaction vessel a helium gas at a rate of 100 mL/min for 30 minutes, a fluorine gas is introduced at a rate of 100 mL/min for 15 minutes. While introducing the fluorine gas at the same rate, a mixture of 4 g (16.1 mmol) of compound (8′) and 4 g of AK-225 are added thereto over 130 minutes. Thereafter, while introducing the fluorine gas at the same rate, a mixture of 0.25 g (1.35 mmol) of hexafluorobenzene and 0.25 g of FC-72 is added thereto over 15 minutes. After introducing a helium gas at a rate of 100 mL/min for 30 minutes, the reaction solution is filtered, and the solvent is distilled off under ordinary pressure to obtain 4.55 g (14.2 mmol; yield: 88.3%) of compound (2′).

Compound (2′): ¹⁹F-NMR [CDCl₃]: δ[ppm]=−89.0 (1F), −86.7 (1F), −74.6 (1F), −73.6 (2F), −72.6 (1F); GC-MS [SCI]: m/z=283 [M⁺-Cl]

### Example 17

### Preparation of Compound (3′)

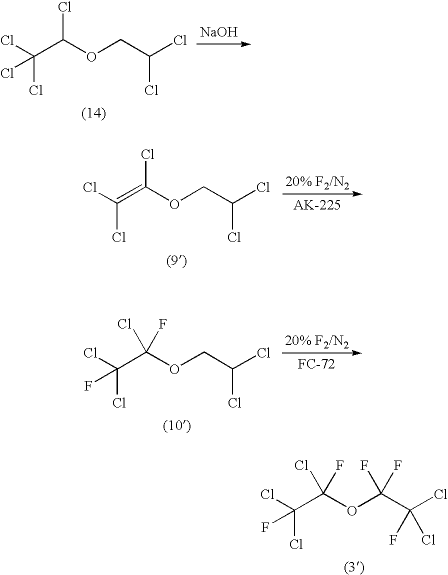

(2′-1) Preparation of Compound (9′)

The same procedures as in (1′-1) of Example 16 are conducted except for changing the compound (10) to 6 g (21.4 mmol) of compound (14) to obtain 4.81 g (19.7 mmol; yield: 92.1%) of compound (9′).

(2′-2) Preparation of Compound (10′)

The same procedures as in (1′-2) of Example 16 are conducted except for changing the compound (7′) to 4.5 g (18.4 mmol) of compound (9′) to obtain 4.71 g (16.7 mmol; yield: 90.9%) of compound (10′).

(2′-3) Preparation of Compound (3′)

The same procedures as in (1′-3) of Example 16 are conducted except for changing the compound (8′) to 4.5 g (15.9 mmol) of compound (10′) to obtain 4.81 g (14.3 mmol; yield: 89.9%) of compound (3′).

Compound (3′): ¹⁹F-NMR [CDCl₃]: δ[ppm]=−87.6 (2F), −76.2 (1F), −74.6 (1F), −72.9 (1F); GC-MS [SCI]: m/z=299 [M⁺-Cl]

### Example 18

### Preparation of Compound (4′)

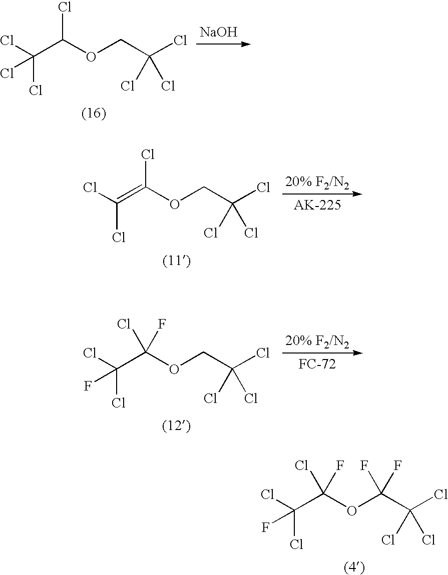

(3′-1) Preparation of Compound (11′)

The same procedures as in (1′-1) of Example 16 are conducted except for changing the compound (10) to 5.5 g (17.4 mmol) of compound (16) to obtain 4.60 g (16.5 mmol; yield: 94.8%) of compound (11′).

(3′-2) Preparation of Compound (12′)

The same procedures as in (1′-2) of Example 16 are conducted except for changing the compound (7′) to 4.5 g (16.1 mmol) of compound (11′) to obtain 4.81 g (15.2 mmol; yield: 94.4%) of compound (12′).

(3′-3) Preparation of Compound (4′)

The same procedures as in (1′-3) of Example 16 are conducted except for changing the compound (8′) to 4.5 g (14.2 mmol) of compound (12′) to obtain 4.76 g (13.5 mmol; yield: 95.1%) of compound (4′).

Compound (4′): ¹⁹F-NMR [CDCl₃]: δ[ppm]=−88.7 (2F), −74.7 (1F), −73.2 (1F); GC-MS [SCI]: m/z=315 [M⁺-Cl]

### Example 19

### Preparation of Compound (5′)

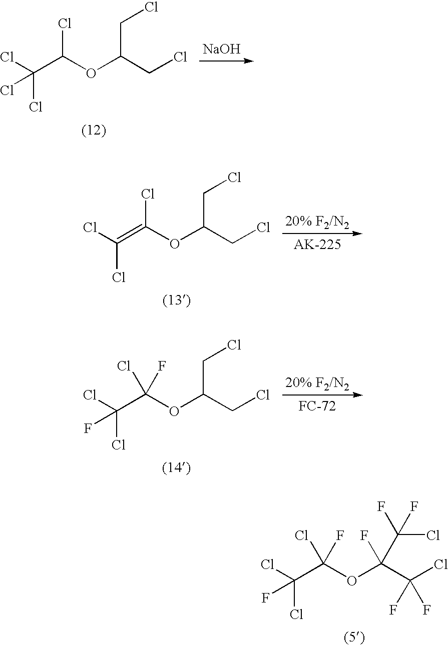

(4′-1) Preparation of Compound (13′)

The same procedures as in (1′-1) of Example 16 are conducted except for changing the compound (10) to 6 g (20.4 mmol) of compound (12) to obtain 5.06 g (19.6 mmol; yield: 96.1%) of compound (13′).

(4′-2) Preparation of Compound (14′)

The same procedures as in (1′-2) of Example 16 are conducted except for changing the compound (7′) to 4.5 g (17.4 mmol) of compound (13′) to obtain 4.95 g (16.7 mmol; yield: 96.0%) of compound (14′).

(4′-3) Preparation of Compound (5′)

The same procedures as in (1′-3) of Example 16 are conducted except for changing the compound (8′) to 4.5 g (15.2 mmol) of compound (14′) to obtain 5.60 g (14.5 mmol; yield: 95.4%) of compound (5′).

Compound (5′): ¹⁹F-NMR [CDCl₃]: δ[ppm]=−136.9 (1F), −64.7 (4F); GC-MS [SCI]: m/z=349 [M⁺-Cl]

### Example 20

### Measurement of Solubility

The same procedures as in Comparative Example 1 are conducted except for changing Halocarbon 1.8 oil to compound (2′). The saturated solubility of the compound (17) in the compound (2′) is found to be 21% by weight.

### Example 21

### Measurement of Solubility

The same procedures as in Example 5 are conducted except for changing the compound (2) to the compound (3′), and it is found that the compound (17) is soluble in the compound (3′) in any proportion.

### Example 22

### Measurement of Solubility

The same procedures as in Example 5 are conducted except for changing the compound (2) to the compound (4′), and it is found that the compound (17) is soluble in the compound (4′) in any proportion.

### Example 23

### Measurement of Solubility

The same procedures as in Comparative Example 1 are conducted except for changing Halocarbon 1.8 oil to the compound (5′). The saturated solubility of the compound (17) in the compound (5′) is found to be 27% by weight.

Also, it can be confirmed that the compound (2′), compound (3′), compound (4′), and compound (5′) used in Examples 20 to 23, respectively, can dissolve FC-72 in any proportion.

Results of measurement of solubility conducted in Examples 20 to 23 can be tabulated in the following Table 2. In Table 2, boiling points and (chlorine content/fluorine content) values of the fluorine-containing solvents are also given.

TABLE 2

| | Boiling | {(Chlorine | | |
|---|---|---|---|---|
| | Point/° C. | Content)/(Fluorine | Solubility (% by weight) | |
| Solvent | 760 mmHg | Content)} Value | Compound (17) | FC-72 |
| Example 20 | 113 | 1.24 | 21 | soluble in any |
| Compound (2′) | | | | proportion |
| Example 21 | 147 | 1.87 | soluble in any | soluble in any |
| Compound (3′) | | | proportion | proportion |
| Example 22 | 185 | 2.80 | soluble in any | soluble in any |
| Compound (4′) | | | proportion | proportion |
| Example 23 | 159 | 1.33 | 27 | soluble in any |
| Compound (5′) | | | | proportion |

### Example 24

### Preparation of Compound (20) by Fluorination of Compound (17) Using Compound (2′) as a Solvent

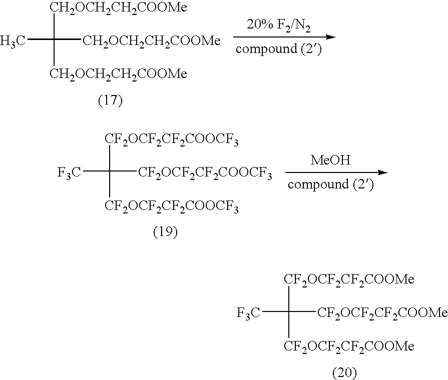

Preparation of the compound (20) is conducted in the same manner as in Example 10 except for changing the solvent from the compound (2) to the compound (2′). The amount of the obtained compound (20) is 1.95 g (2.58 mmol; yield: 97.7%), and the GC purity thereof is 89%.

### Example 25

### Preparation of Compound (20) by Fluorination of Compound (17) Using Compound (5′) as a Solvent

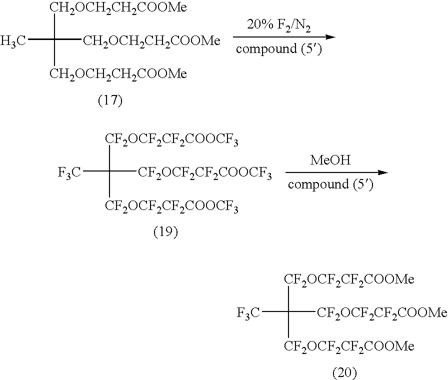

Preparation of the compound (20) is conducted in the same manner as in Example 10 except for changing the solvent from the compound (2) to the compound (5′). The amount of the obtained compound (20) is 1.96 g (2.59 mmol; yield: 98.1%), and the GC purity thereof is 88%.

### Example 26

### Preparation of Coating Film Using a Composition Containing a Fluorine-Containing Acrylate and a Fluorine-Free Acrylate

20 parts by weight of trimethylolpropane triacrylate [manufactured by Tokyo Chemical Industry Co., Ltd.; fluorine content: 0% by weight], 20 parts by weight of 1,6-bis(acryloyloxy)-2,2,3,3,4,4,5,5-octafluorohexane [manufactured by Tokyo Chemical Industry Co., Ltd.; fluorine content: 41%], 4 parts by weight of a photo initiator of Irgacure 184 (manufactured by Ciba-Geigy Ltd.; fluorine content: 0% by weight), and 80 parts by weight of a solvent of the compound (2′) are mixed to prepare a uniform curable composition. This composition is spin-coated on a 100-μm thick PET film. Then, curing is conducted by irradiating 400-mJ/cm² UV rays using a UV ray-irradiating apparatus having a 120-W high-pressure mercury lamp to thereby prepare a PET film having a uniform hard coat film formed on the surface thereof.

### Comparative Example 7

The same procedures as in Example 26 are conducted except for changing the compound (2′) to FC-72. Trimethylolpropane triacrylate is insoluble in FC-72, and hence a uniform hard coat film is not obtained.

As has been described above, use of the perfluorochloroether of the invention serves to enhance solubility of both the fluorine-free organic compound and the fluorine-containing organic compound, thus a uniform coating film being obtainable.

As is apparent from above Examples, various organic compounds can be dissolved, regardless of the fluorine content (from 0 to 79% by weight) and kinds of functional groups (hydrocarbon group, ether group, ester group, carbonyl group, hydroxyl group, aryl group, isocyanato group, urethane group, acrylate group, trialkoxysilyl group, siloxy group, etc.) and regardless of whether the compounds are liquids or solids, by using the perfluorochloroether solvent of the invention. Thus, it can be seen that, in order to dissolve these organic compounds, it is of extreme importance to use a perfluorochloroether solvent having the (chlorine content/fluorine content) value in the range of from 1.2 to 4.5. Therefore, organic compounds which can be dissolved in the perfluorochloroether solvent of the invention are not particularly limited, and organic compounds having various fluorine contents and various structures can be used.

According to the present invention, there can be provided a liquid composition containing a perfluorochloroether solvent having an extremely high dissolution capacity for both a fluorine-free organic compound and a fluorine-containing compound. The composition is made extremely useful as, for example, a chemical reaction solution, a detergent, a coating agent, or a coolant by further dissolving or dispersing therein other appropriate effective ingredients.

While the invention has been described in detail and with reference to specific embodiments thereof, it will be apparent to one skilled in the art that various changes and modifications can be made therein without departing from the spirit and scope of the present invention.

This application is based on Japanese Patent Application (Japanese Patent Application No. 2009-083789) filed on Mar. 30, 2009 and Japanese Patent Application (Japanese Patent Application No. 2010-076452) filed on Mar. 29, 2010, the contents of which are incorporated herein by way of reference.

What is claimed is:

1. A liquid composition comprising:
a perfluorochloroether solvent that contains a perfluorochloroether compound having a (chlorine content/fluorine content) value within the range of from 1.2 to 4.5; and
an organic compound that has a fluorine content of from 0 to 79% by weight and that is dissolved in the perfluorochloroether solvent,
wherein the (chlorine content/fluorine content) value is a value defined by the following formula:
(chlorine content/fluorine content)value=(number of chlorine atoms in perfluorochloroether×atomic weight of chlorine atom)/(number of fluorine atoms in perfluorochloroether×atomic weight of fluorine atom).

2. The liquid composition according to claim 1, wherein the (chlorine content/fluorine content) value of the perfluorochloroether compound is within the range of 1.8 to 4.5.

3. The liquid composition according to claim 1, wherein
the perfluorochloroether solvent has a boiling point within the range of from 60° C. to 300° C. at a pressure of 760 mmHg.

4. The liquid composition according to claim 1, wherein
the organic compound having a fluorine content of from 0 to 79% by weight is a polymerizable compound.

5. The liquid composition according to claim 1, wherein
the organic compound having a fluorine content of from 0 to 79% by weight is a polymer.

6. The liquid composition according to claim 1, wherein
the perfluorochloroether compound is a compound represented by the following general formula (1): 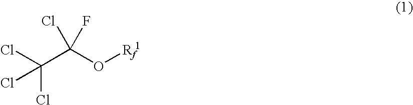
wherein
R_{f}¹ represents a hydrocarbon group, all the hydrogen atoms of which are replaced by a fluorine atom or a chlorine atom, provided that the hydrocarbon group may contain an etheric oxygen atom.

7. The liquid composition according to claim 6, wherein
the compound represented by the foregoing general formula (1) is a compound represented by the following formula (2), (3), (4) or (5):

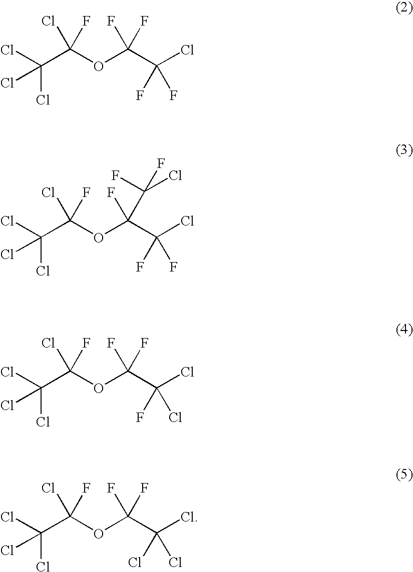

8. The liquid composition according to claim 1, wherein
the perfluorochloroether compound is a compound represented by the following general formula (1′): 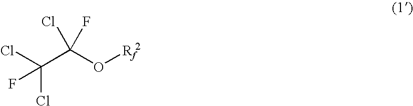
wherein
R_{f}² represents a hydrocarbon group, all the hydrogen atoms of which are replaced by a fluorine atom or a chlorine atom, provided that the hydrocarbon group may contain an etheric oxygen atom.

9. The liquid composition according to claim 8, wherein
the compound represented by the foregoing general formula (1′) is a compound represented by the following formula (2′), (3′), (4′) or (5′):

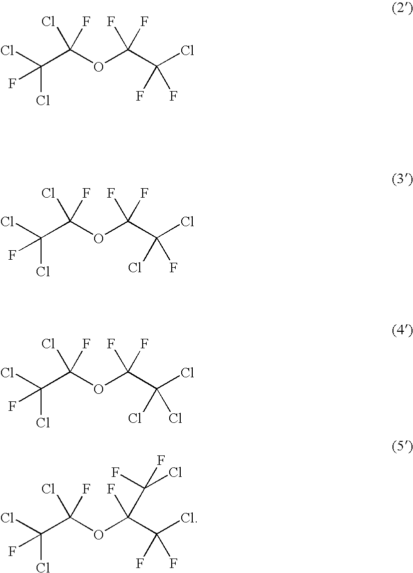

10. A liquid composition for coating, which comprises the liquid composition described in claim 1.

11. A chemical reaction solution which comprises the liquid composition described in claim 1.

12. A perfluorochloroether compound represented by the following formula (2):

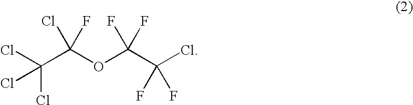

13. A perfluorochloroether compound represented by the following formula (3):

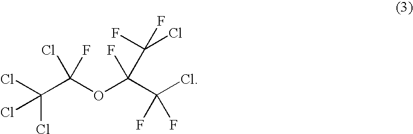

14. A perfluorochloroether compound represented by the following formula (4):

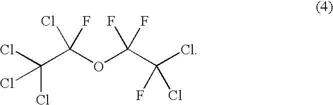

15. A perfluorochloroether compound represented by the following formula (5):

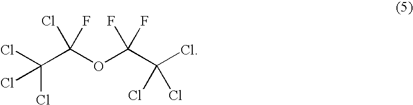

16. A perfluorochloroether compound represented by the following formula (2′):

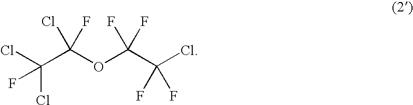

17. A perfluorochloroether compound represented by the following formula (3′):

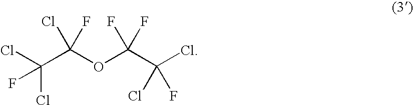

18. A perfluorochloroether compound represented by the following formula (4′):

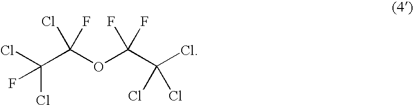

19. A perfluorochloroether compound represented by the following formula (5′): [chemical structure]
